(19) United States
(12) Patent Application Publication
(10) Pub. No.: US 2009/0081145 A1
(43) Pub. Date: Mar. 26, 2009

(54) PROCESS FOR FORMING DISULPHIDE BRIDGES

(75) Inventors: Karsten Knorr, Krefeld (DE); Marco Emgenbroich, Neuss (DE); Carsten Bungener, Baesweiler (DE)

Correspondence Address: HENRY M FEIEREISEN, LLC;HENRY M FEIEREISEN, 708 THIRD AVENUE, SUITE 1501, NEW YORK, NY, 10017, US

(73) Assignee: AplaGen GmbH, Baesweiler (DE)

(21) Appl. No.: 12/158,875

(22) PCT Filed: Dec. 23, 2006
(86) PCT No.: PCT/EP06/12526

§ 371 Date: Sep. 17, 2008

(30) Foreign Application Priority Data
Dec. 23, 2005 (EP) 05028311.8
Mar. 16, 2006 (EP) 06005438.4
Nov. 17, 2006 (EP) 06023953.0

(51) Int. Cl. A61K 8/49; C07K 14/47; C07K 7/00; C07K 1/08; C07D 239/48; C07D 487/04; C07D 239/54; C07D 475/00; A61Q 5/00

(52) U.S. Cl. 424/70.1; 530/350; 530/330; 530/329; 530/328; 530/327; 530/326; 530/325; 530/324; 544/323; 544/262; 544/309; 544/257

(57) ABSTRACT

The present invention relates to an improved method of formation of disulfide bridges in substances bearing SH groups, in particular peptides, for example by formation of intramolecular disulfide bridges, in which a heterocyclic compound having at least one nitrogen atom (e.g. caffeine or a caffeine-like substance) is used for catalysis of the reaction. It was found, surprisingly, that addition of the heterocyclic substance increases both the yield and the purity of the product bearing disulfide bridges.

Various methods of formation of disulfide bridges are known in the prior art. For example, the use of K₃[Fe(CN)₆] as oxidizing agent is known in one standard method of cyclization of peptides by formation of intramolecular disulfide bridges. This reagent provides clean cyclization products at high yields; side reactions are avoided. Furthermore, cyclizations under the influence of oxygen or iodine are also known. These methods have the disadvantage, however, that they are either too slow, or they lead to a large number of undesirable by-products. Another known method of cyclization of peptides is the use of immobilized Ellmann's reagent (5.5′-dithiobis(2-nitrobenzoic acid)) as oxidizing agent. This method makes possible the complete oxidation of e.g. a linear peptide; contamination by the reagent is avoided. This approach was extended just recently to crosslinked ethoxylate-acrylate resin (CLEAR) supports. These are compatible both with organic and with aqueous solvent mixtures.

Another method known in the prior art for formation of disulfide bridges e.g. for the cyclization of peptides is the use of DMSO as oxidizing agent. This process also leads to complete oxidation of the linear peptide. This method has the disadvantage, however, that the reaction takes place very slowly and that the excess of DMSO must be removed prior to further processing, which is difficult with this organic solvent.

It can be seen from the above that several methods are known for forming disulfide bridges, which is important in particular in the cyclization of peptides through formation of intramolecular disulfide bridges. However, each of these methods has disadvantages, either with respect to yield, or reaction rate, or purity.

The present invention is therefore based on the problem of providing an alternative method for the production of disulfide bridges.

This problem is solved by a method of formation of disulfide bridges that is characterized in that the reaction is carried out in a liquid mixture, which contains at least one heterocyclic compound having at least one nitrogen atom in the ring.

Accordingly, claim 1 relates to a method of formation of disulfide bridges, which is characterized in that the reaction is carried out in a liquid medium that contains at least one compound which promotes the formation of disulfide bridges, said compound being selected from the following group:

a compound having in its structure a saturated or unsaturated six-membered heterocycle with at least one nitrogen atom, said heterocycle having at least one hydroxyl group or an oxo group (═O) according to the invention on the carbon atom adjacent to the nitrogen atom, and if a hydroxyl group is present the heterocycle is unsaturated;
a compound of the following general formula

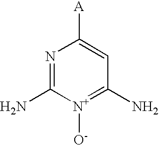

in which substituent A stands for
hydrogen, an optionally substituted alkyl residue, an optionally substituted aryl residue or a saturated or unsaturated heterocyclyl with 3 to 10 ring members and 1 to 3 heteroatoms, such as nitrogen, oxygen and/or sulfur, the heterocyclyl being unsubstituted or substituted one or more times with halogen, alkyl with 1 to 4 carbon atoms, cyano, nitro, cycloalkyl with 3 to 6 carbon atoms, hydroxy, alkoxy with 1 to 4 carbon atoms and/or mercapto.

It was found, surprisingly, that the compounds defined above promote the formation of disulfide bridges and therefore can act as a kind of catalyst in the reaction. It is therefore advantageous to add these compounds to the reaction mixture, in order to promote the formation of disulfide bridges in various substances bearing SH groups, such as in particular peptides and proteins.

According to one embodiment the compound according to alternative (a) has the following basic structure:

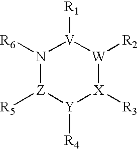

where, depending on the choice of the substituents R1 to R6, the heterocycle is saturated or unsaturated and accordingly can have one or more double bonds;
V, W, X, Y and Z represent either carbon atoms or nitrogen atoms, the heterocycle having a total of not more than three, preferably two, nitrogen atoms;
R1 represents either a hydroxyl group or an oxo group (═O) according to the invention;
R2 and R3, always independently of one another, stand for hydrogen, an optionally substituted alkyl or aryl residue, an optionally substituted residue —(CH₂)ₙCOOX with n equal to 0 to 10 and X equal to hydrogen or alkyl, an electron-withdrawing substituent, a functional group such as in particular a hydroxyl group, an oxo group (═O) according to the invention, —CONH₂ or an oxime (═N—OH) or R2 together with R3 represents a five- or six-membered ring, which can also have heteroatoms and optionally carries other substituents, or R2 and/or R3 are absent;
R4 and R5, always independently of one another, stand for hydrogen, an optionally substituted alkyl or aryl residue, an electron-withdrawing substituent, a functional group such as in particular a hydroxyl group, an oxo group (═O) according to the invention, a carboxyl group, —CONH₂ or an oxime (═N—OH) or R4 together with R5 represents a five- or six-membered ring, which can also have heteroatoms and optionally carries other substituents, R5 together with R6 represents a five- or six-membered ring, which can also have heteroatoms and optionally carries other substituents or R4 and/or R5 are absent;

R6 stands for hydrogen or an optionally substituted alkyl or aryl residue, or R6 together with R5 represents a five- or six-membered ring, which can also have heteroatoms and optionally carries other substituents, or R6 is absent.

Said substituents R2 to R6 can be absent when a nitrogen atom that has a double bond is located at the linking position in the ring (see e.g. the compounds 2,6-dihydroxypyridine hydrochloride; uracil-6-carboxylic acid, 4,6-dihydroxypyrimidine).

A central feature of the substances of the invention according to alternative (a) is the presence of the six-membered, nitrogen-containing heterocycle and the hydroxyl group or oxo group (═O) according to the invention on the adjacent carbon atom. “Oxo group” in the sense of the invention means that the particular substituent together with the ring atom forms an oxo group and accordingly an oxygen atom is bound to the ring via a double bond:

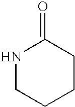

Thus, extensive tests have shown that compounds without such a functional group (hydroxyl group, oxo group ═O) regularly do not have the capacity to promote the formation of disulfide bridges (for example aminopyrazine, 2,4-diaminopyrimidine, melamine, pyrazine-carboxylic acid and pyrazine amide).

As follows from the definition of the substituents, there are compounds that have only one nitrogen atom in the heterocycle and display advantageous effects with respect to promoting the formation of disulfide bonds. An example of a compound with only one nitrogen atom in the heterocycle and an oxo group (═O) according to the invention on the adjacent carbon atom is the compound N-methyl-2-pyridone:

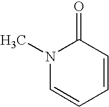

Another example of a compound with only one nitrogen atom in the heterocycle and with a hydroxyl group on the adjacent carbon atom is the compound 2,6-dihydroxy-pyridine hydrochloride:

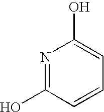

As stated in the claim, in the case when hydroxyl groups are present the heterocycle is unsaturated. Tests have shown that in this case the presence of at least one double bond is apparently important for the catalytic action. Without wishing to be tied to this, it is speculated that this might be attributable to the fact that compounds of this structure can tautomerize. Tautomers are structural isomers that only differ in the position of a group (e.g. hydrogen) and in the position of a double bond. In the case of the hydroxyl group and the oxo group (═O) we also talk of keto-enol tautomerism. It has proved advantageous if compounds in aromatic form, such as 2,6-dihydroxy-pyridine hydrochloride, can tautomerize. This applies in particular to the enol forms, which can preferably tautomerize in the direction of the keto form. Tautomeric (isomeric) forms of the stated substances are therefore included according to the invention.

The six-membered heterocycle preferably has two nitrogen atoms, which can be in different positions. Examples of active compounds of this structure are uracil-6-carboxylic acid, 2,4-dihydroxy-6-methylpyrimidine, 2,4-dimethyl-6-hydroxypyrimidine, 2-isopropyl-6-methyl-4-pyrimidinol, 4,6-dihydroxy-2-methylpyrimidine, 4,6-dihydroxy-pyrimidine, 1,2-dihydro-3,6-pyridazinedione:

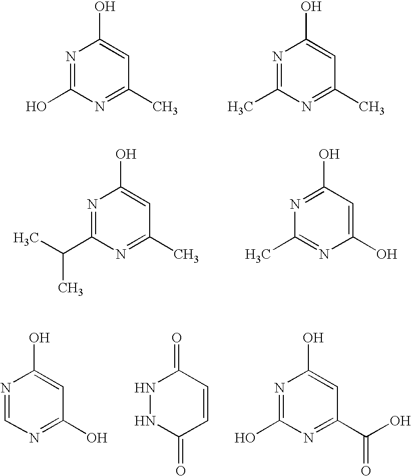

An example of compounds in which R5 and R6 together form a ring structure is 7-hydroxy-5-methyl1.2.4]triazolo[1,5-a]pyrimidine:

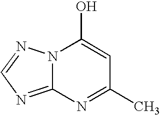

According to a further embodiment the compound has the following substructure:

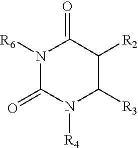

and, depending on the choice of the substituents R2, R3, R4 and R6, the heterocycle is saturated or unsaturated and accordingly can have one or more double bonds; where
R2 and R3, always independently of one another, stand for hydrogen, an optionally substituted alkyl or aryl residue, an optionally substituted residue —(CH₂)ₙCOOX with n equal to 0 to 10 and X equal to hydrogen or alkyl, an electron-withdrawing substituent, a functional group such as in particular a hydroxyl group, an oxo group (═O) according to the invention, —CONH₂ or an oxime (═N—OH) or R2 together with R3 represents a five- or six-membered ring, which can also have heteroatoms and optionally carries other substituents, or R2 and/or R3 are absent;
R4 stands for hydrogen or an optionally substituted alkyl or aryl residue or R4 is absent;
R6 stands for hydrogen or an optionally substituted alkyl or aryl residue or R6 is absent.

Active examples of this embodiment are e.g. barbituric acid, alloxan monohydrate and violuric acid

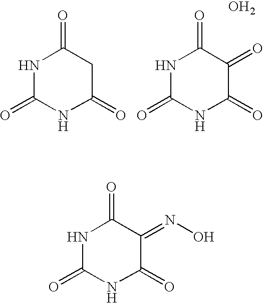

Further examples of this embodiment are uracil derivatives with the following general formula:

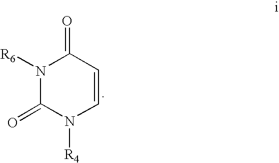

in which R4 and R6, independently of one another, stand for hydrogen or an optionally substituted alkyl or aryl residue, preferably for hydrogen or a linear or branched C1 to C10 alkyl residue, especially preferably for a C1 to C4 alkyl residue or hydrogen.

Examples with good activity are uracil and 1-methyl-uracil:

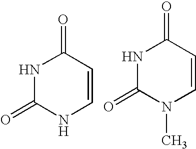

According to a further embodiment, the compound that promotes the formation of disulfide bonds comprises purine derivatives, whose basic structure corresponds to the following general formula

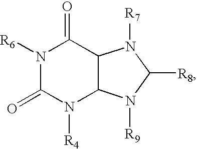

where the five-membered ring is unsaturated and accordingly has double bonds and where
R4 and R6, always independently of one another, stand for hydrogen, an optionally substituted alkyl residue or an optionally substituted aryl residue; preferably hydrogen, an optionally substituted C₁ to C₁₀ alkyl residue or an optionally substituted C₆ or C₁₀ aryl residue; especially preferably hydrogen, an optionally substituted C₁ to C₆ alkyl residue or an optionally substituted C₆ aryl residue; in particular for hydrogen or an optionally substituted C₁ to C₃ alkyl residue;
R7, R8 and R9, always independently of one another, stand for hydrogen, an optionally substituted alkyl residue, an optionally substituted aryl residue or an optionally substituted residue —(CH₂)ₙCOOX with n equal to 0 to 10 and X equal to hydrogen or alkyl or a functional group; preferably hydrogen, an optionally substituted C₁ to C₁₀ alkyl residue, an optionally substituted C₆ or C₁₀ aryl residue or an optionally substituted residue —(CH₂)ₙ—COOX with n=1 to 10 and X equal to hydrogen or C₁ to C₈ alkyl; especially preferably hydrogen, an optionally substituted C₁ to C₆ alkyl residue, an optionally substituted C₆ aryl residue or an optionally substituted residue —(CH₂)ₙ—COOX with n=1 to 6 and X equal to hydrogen or C₁ to C₆ alkyl; in particular hydrogen, an optionally substituted C₁ to C₃ alkyl residue or an optionally substituted residue —(CH₂)ₙ—COOX with n=1 to 4 and X equal to hydrogen or C₁ to C₃ alkyl.

As shown above, the cyclic compound can accordingly be based on a purine basic structure. The purine basic structure can be thought of as a condensed ring system, made up of the two heterocycles pyrimidine and imidazole. Its systematic IUPAC name is 7H-imidazole[4,5-d]pyrimidine.

7H-purine is in tautomeric equilibrium with its isomer 9H-purine, and compounds that are based on both tautomeric forms are also considered to be within the scope of the present invention:

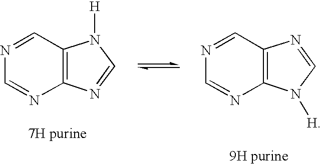

Depending on the choice of substituents on the pyrimidine ring, the latter can also have fewer double bonds.

In a quite especially preferred variant of the present invention at least one residue R4, R6, R7 and R9 is an alkyl group and at least one, preferably two of the residues R4, R6, R7 and R9 represent hydrogen or a C₁ to C₃ alkyl group, preferably methyl.

Special examples of these heterocyclic compounds based on purine are 3-methylxanthine, theobromine, theophylline, caffeine, isocaffeine, xanthine, theophylline-7-acetic acid, theophylline-8-butyric acid and 3-isobutyl-1-methylxanthine:

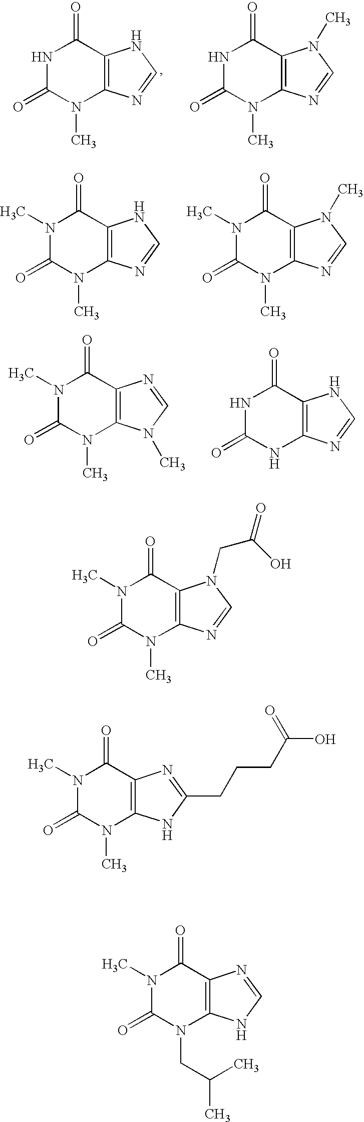

As these selected examples show, tautomeric compounds based on the 9H-purine basic structure instead of the 7H-purine basic structure shown above, are also included.

According to a further embodiment R2 and R3 together form an optionally substituted six-membered ring, optionally having at least one heteroatom. This embodiment relates primarily to compounds in which an aromatic compound was condensed onto the basic heterocycle, preferably with a bridge of two carbon atoms. Examples in which a five-membered ring was condensed on (e.g. imidazole), were discussed above (compounds based on the purine basic structure). Further examples of condensed-on ring structures are pyrazine and quinoxaline.

An example of a compound with a benzene ring as condensed-on six-membered ring is 1,2,3-benzotriazin-4(3H)-one:

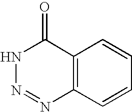

According to an advantageous embodiment the compound has the following basic structure:

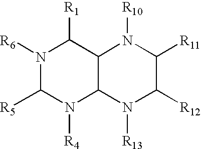

where, depending on the choice of substituents, the rings can be unsaturated and accordingly can have one or more double bonds and
R1 represents either a hydroxyl group or an oxo group (═O) according to the invention;
R4 and R6, always independently of one another, stand for hydrogen, an optionally substituted alkyl residue or an optionally substituted aryl residue or are absent; preferably stand for hydrogen, an optionally substituted linear or branched C₁ to C₁₀ alkyl residue or an optionally substituted C₆ or C₁₀ aryl residue; especially preferably stand for hydrogen, an optionally substituted C₁ to C₆ alkyl residue or an optionally substituted C₆ aryl residue; in particular hydrogen or an optionally substituted C₁ to C₃ alkyl residue;
R5 stands for hydrogen, an optionally substituted alkyl or aryl residue, an electron-withdrawing substituent, a functional group such as in particular a hydroxyl group or an oxo group (═O) according to the invention, or R5 is absent;
R10 and R13, always independently of one another, stand for hydrogen, an optionally substituted alkyl residue or an optionally substituted aryl residue, or R10 and/or R13 are absent; preferably stand for hydrogen, an optionally substituted linear or branched C₁ to C₁₀ alkyl residue or an optionally substituted C₆ or C₁₀ aryl residue; especially preferably stand for hydrogen, an optionally substituted C₁ to C₆ alkyl residue or an optionally substituted C₆ aryl residue; in particular for hydrogen or a C₁ to C₆ alkyl residue substituted with at least one hydroxyl group;
R11 and R12, always independently of one another, stand for hydrogen, an optionally substituted alkyl or aryl residue, an electron-withdrawing substituent, a functional group such as a hydroxyl group, an oxo group (═O) according to the invention, a carboxyl group, —CONH₂ or an oxime (═N—OH) or R11 and R12 together form a five or six-membered ring, which can optionally have further heteroatoms and substituents.

Selected examples of compounds covered by this formula are e.g. (−)-riboflavin, lumazin and alloxazin

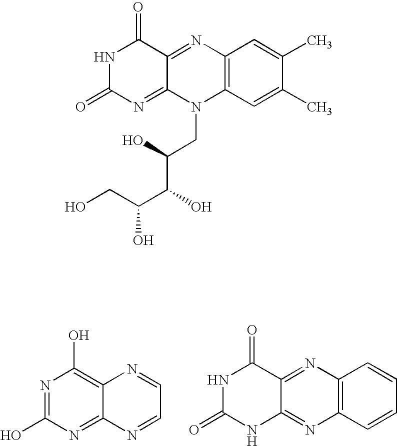

It has proved advantageous for the action if the compounds of the invention according to alternative (a) do not have any exocyclic amino groups. Preferably, therefore, the compounds that are to be used according to the invention do not have any exocyclic amino groups.

The aforementioned substituents R2, R3, R4 and R5 can moreover, independently of one another, be hydrogen, an optionally substituted linear or branched C₁ to C₁₀ alkyl residue or an optionally substituted C₆ or C₁₀ aryl residue; preferably hydrogen, an optionally substituted linear or branched C₁ to C₆ alkyl residue or an optionally substituted C₆ aryl residue; in particular hydrogen or an optionally substituted C₁ to C₃ alkyl residue, in particular methyl residue.

R6 is preferably hydrogen or an alkyl residue, C₁ to C₈, preferably C₁ to C₄, especially preferably hydrogen or a methyl group.

Electron-withdrawing groups or atoms, which can also be used as substituents on the heterocycle (see above), are e.g. electron-withdrawing groups or atoms which, as substituents, lower the electron density on a corresponding aromatic heterocyclic ring (also called deactivating groups). Electron-withdrawing groups possess an (−)-M- and/or an (−)-I-effect. The resonance effect (M-effect mesomeric effect) is generally only operative when the group is bound directly to the unsaturated heterocyclic system. It operates via π-electrons, in contrast to the field effect (I-effect, inductive effect), which operates via space, via solvent molecules or preferably via σ-bonds of a system.

An electron-withdrawing effect can take place either inductively (i.e. by the so-called (−)-I-effect) and/or mesomerically (i.e. by the so-called (−)-M-effect). The division of aromatic substituents into substituents with (+)-I- and (−)-I-effect and with (+)-M-effect and (−)-M-effect is already familiar to a person skilled in the art. For further details reference should be made to Beyer/Walter, “Lehrbuch der organischen Chemie” [Textbook of organic chemistry], 1998, 23rd revised and updated edition, pages 515 to 518, the relevant disclosure of which is included in the present invention.

Some nonlimiting examples of groups with (−)-M-effect are —SO₂—, —SO₂O—, —OO—, —COO—, —CONH—, —CONR—, —SOR—, —CN, —NO₂, —CHO, —CO—, —COSH, —COS⁻, —SO₃H and the oxo group (═O) according to the invention. As is apparent, the terms “electron-withdrawing group” and “functional group” can overlap. Corresponding groups are examples of substituents that can be used.

According to a further embodiment, the compound according to alternative (b) has a substituent A that stands for
hydrogen, an optionally substituted C₁ to C₁₀ alkyl residue, an optionally substituted C₆ or C₁₀ aryl residue or a saturated or unsaturated heterocyclyl with 3 to 10 ring members and 1 heteroatom, such as nitrogen, oxygen and/or sulfur, the heterocyclyl being unsubstituted or substituted one or more times with halogen, alkyl with 1 to 4 carbon atoms, cyano, nitro, cycloalkyl with 3 to 6 carbon atoms, hydroxy, alkoxy with 1 to 4 carbon atoms and/or mercapto;
preferably hydrogen, an optionally substituted C₁ to C₆ alkyl residue, an optionally substituted C₆ aryl residue or saturated heterocyclyl with 5 or 6 ring members and 1 heteroatom, such as nitrogen, oxygen and/or sulfur, the heterocyclyl being unsubstituted or substituted one or more times with halogen, alkyl with 1 to 4 carbon atoms, cyano, nitro, cycloalkyl with 3 to 6 carbon atoms, hydroxy, alkoxy with 1 to 4 carbon atoms and/or mercapto;
in particular hydrogen, an optionally substituted C₁ to C₃ alkyl residue or saturated heterocyclyl with 5 or 6 ring members and 1 heteroatom, such as nitrogen, oxygen and/or sulfur, the heterocyclyl being unsubstituted.

Special examples of these pyrimidine derivatives are the following compounds:

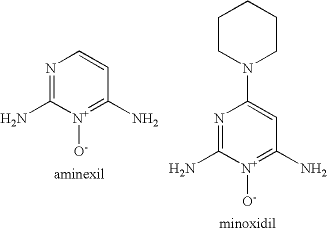

Functional groups on the heterocyclic compound that is to be used according to the invention are helpful for facilitating binding of the substance to the support. Therefore it is also possible for other derivatives of heterocyclic compounds not previously mentioned explicitly to be used according to the invention.

The heterocyclic compounds described above can be both in pure form and as mixtures of various possible isomeric forms, in particular of stereoisomers, such as E- and Z-, threo- and erythro-, and optical isomers, such as R- and S-isomers or atropisomers, and of tautomers. The invention includes both the pure isomers and mixtures thereof.

Depending on the type of substituents defined above, the heterocyclic compounds have acid or basic properties and can form salts, optionally also internal salts. If the compounds of formula (I) bear hydroxyl, carboxyl or other groups that give rise to acid properties, these compounds can be reacted with bases to form salts. Suitable bases are for example hydroxides, carbonates, hydrogencarbonates of the alkali metals and alkaline-earth metals, in particular those of sodium, potassium, magnesium and calcium, in addition ammonia, primary, secondary and tertiary amines with (C₁-C₄)-alkyl residues and mono-, di- and trialkanolamines of (C₁-C₄)-alkanols. If the compounds of formula (I) bear amino, alkylamino or other groups that give rise to basic properties, these compounds can be reacted with acids to form salts. Suitable acids are for example mineral acids, such as hydrochloric, sulfuric and phosphoric acid, organic acids, such as acetic acid or oxalic acid, and acid salts, such as NaHSO₄ and KHSO₄. The salts obtainable in this way can also be used.

Further preferred groups of the aforementioned compounds will be discussed later.

Examples of heterocyclic compounds that can be used according to the invention for promoting the formation of disulfide bridges can be described further as follows:

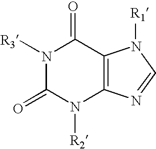

The residues R₁′, R₂′ and R₃′ are either identical or different; however, at least one of the residues is an alkyl group. Of course, tautomers in which, among other things, double bond shift occurs, are also covered by the above formula. Corresponding structural isomers are therefore also covered by this formula. These compounds are, as explained, suitable in particular for promoting the formation of disulfide bridges in amino acid-containing substances, in particular in peptides and proteins.

At least one, preferably two of the residues R₁′, R₂′ and R₃′, which are either identical or different, preferably represent either hydrogen or a C₁ to C₅ alkyl group. In particular, short-chain alkyl groups with 1 to 3 carbon atoms, in particular the methyl group, have proved advantageous. Moreover, at least one of the residues can contain a functional group. Moreover, it is also possible for residues R₄′ (on the nitrogen) and R₅′ (on the carbon located between the nitrogen atoms) to be present in the remaining positions on the heterocyclic five-membered ring (in particular in the case of the tautomeric forms). These are residues of any form, preferably organic residues. According to one embodiment they are functional groups that make it possible for the substance to bind e.g. to a support. This variant will be described in more detail later.

Especially suitable substances for use with the method according to the invention are, as already stated, both caffeine and caffeine-like substances, for example theobromine and theophylline.

It was found, surprisingly, that peptides and proteins in particular, especially peptides with an amino acid length between 5 and 100, preferably 10 and 50, especially preferably between 15 to 40 amino acids can be cyclized in water even at higher peptide concentration at room temperature by the method according to the invention through the formation of intramolecular disulfide bridges. The method is therefore especially suitable for the formation of intramolecular disulfide bridges and therefore in particular for the cyclization of peptides. Moreover, polypeptides and proteins can be cyclized with the corresponding method. In addition, disulfide bridges can also be formed in substances with other structures, bearing SH groups. The formation of disulfide bridges, e.g. during cyclization, takes place almost quantitatively in some peptides based on addition of the heterocyclic compound according to the invention, e.g. caffeine or a caffeine-like substance (see above formulas). However, this is not absolutely necessary. Surprisingly, it is not necessary (though possible) to add an oxidizing agent to speed up the reaction, because in the presence of the substance characterized above, the oxygen of the air is sufficient for the formation of disulfide bridges.

Test results have shown, moreover, that with the method according to the invention the reaction rate can increase in the course of the reaction. The positive effect from addition, according to the invention, of the substance characterized above is surprising, since—in contrast to the substances DMSO or iodine used in the prior art—it is not generally an oxidizing agent. In the method according to the invention the oxygen of the air is sufficient for oxidation, and there is an advantageous increase in reaction rate as a result of addition of the heterocyclic compound according to the invention. The increase in reaction rate might also be due to an autocatalytic mechanism, possibly by the cyclized product.

Advantageously it has been found that despite the increase in reaction rate and even when using high peptide concentrations, often there is little if any formation of oligomerization products. The formation of intermolecular disulfide bridges, which is undesirable in the case of cyclization of a peptide, was therefore not observed. The peptide concentration to be used depends, however, on the particular peptide used and should therefore be optimized for each peptide.

The method according to the invention can be carried out advantageously at room temperature.

The apparently autocatalytic course of the reaction occurs both in unbuffered and in buffered solutions (e.g. phosphate buffer, pH 6-9). It was found, however, that the reaction rate can be increased considerably if the pH value is lowered. It is therefore advantageous to adjust the pH value to <=7, preferably in a pH range from approx. 4 or 5 to 6.5, and a pH value around 6 (5.5 to 6.5) is especially preferred.

The amount of substance that is added to the reaction mixture in order to promote the formation of in particular intramolecular disulfide bridges varies depending on the compound and the material in which the disulfide bridges are to be formed. As a rule small catalytic amounts are sufficient. The amount to be used is preferably at least approx. 0.0001 mg/ml, especially preferably in a range from approx. 0.0001, 0.001 or 0.01 to 20 mg/ml, 0.001 or 0.01 to 15 mg/ml, 0.001 or 0.01 to 10 mg/ml, 0.001 or 0.01 to 5 mg/ml, preferably 0.001 or 0.01 to 1 mg/ml and especially preferably in a range from 0.03 to 0.5 mg/ml. The amounts vary depending on substance selected (e.g. caffeine or caffeine-like substance) and the peptide or protein to be treated, and should therefore be optimized individually in each case. An especially suitable concentration range for peptides with a length of approx. 15 to 25 amino acids (especially in the case of EPO mimetic peptides) is 0.05 to 0.3 mg/ml, especially preferably 0.075 to 0.15 mg/ml. Once again, however, the amounts vary depending on the peptide and can even be much higher; the amounts should therefore preferably be optimized for the particular peptide.

The reaction rate can be further accelerated if an additional oxidizing agent is added to the reaction mixture. We may mention, for example, glutathione in oxidized form (GSSG).

It was found, surprisingly, that cyclization can also be carried out effectively at high peptide concentrations, without undesirable oligomerizations occurring. For many peptides, high peptide concentrations are therefore not a problem in the method according to the invention. In fact for some peptides it was found later that in the method according to the invention the cyclization reaction proceeds even better at high peptide concentrations. Depending on the peptide, suitable peptide concentrations are approx. 0.05 or 0.1 or 0.5 to 5 mg/ml, and concentrations in a range from 0.7 to 1.5 mg/ml are preferred. The precise concentration depends of course on the particular peptide, its length and its amino acid composition, and varies accordingly. The present details are therefore not to be regarded as limiting. For EPO mimetic peptides it proved especially advantageous to use a concentration from approx. 0.7 to 1 mg/ml. They can be cyclized particularly effectively with the addition of caffeine.

Usually a disulfide bridge is formed in a peptide or protein between two cysteines. However, according to the invention, the disulfide bridge can also be formed between other natural and nonnatural amino acids, if these have corresponding groups that are suitable for the formation of a disulfide bridge (—S—S—). Thiolysine, homocysteine and other cysteine derivatives may be mentioned, along with cysteine, as examples of suitable amino acids. The term disulfide bridge should not, however, be equated with the term cysteine bridge, but comprises the formation of corresponding —S—S— bonds between any natural or nonnatural SH-containing amino acids or other compounds containing SH groups. With the method according to the invention it is therefore also possible to form disulfide bridges in other, in particular polymeric, compounds containing SH groups. With the method according to the invention it is, of course, also possible for several disulfide bridges to be formed.

Especially advantageously, the present method can be used for the cyclization of EPO mimetic peptides (see e.g. WO 96/40479). Novel EPO mimetic peptides are described in PCT/EP2005/012075 (WO 2006/050959), whose disclosure with respect to peptides is hereby incorporated in its entirety in this application. As described in detail in PCT/EP2005/012075 (WO 2006/050959), these novel EPO mimetic peptides do not have proline in position 10 of the EPO mimetic consensus motif (regarding the numbering, see Johnson et al., 1997). Rather, the proline is replaced with a nonconservative amino acid, in particular a basic amino acid such as in particular lysine.

The peptides described in PCT/EP2005/012075 (WO 2006/050959) preferably have the following consensus:

X₆X₇X₈X₉X₁₀X₁₁X₁₂X₁₃X₁₄X₁₅

where each amino acid represents a natural or nonnatural amino acid and
X₆ is C, A, E, a-amino-γ-bromobutyric acid or homocysteine (Hoc);

### X₇ is R, H, L, W or Y or S;

X₈ is M, F, I, homoserine methyl ether (Hsm) or norisoleucine;
X₉ is G or a conservative substitution of G;
X₁₀ is a nonconservative substitution of proline;
or X₉ and X₁₀ are substituted with a single amino acid;
X₁₁ can be any amino acid;

### X₁₂ is T or A;

X₁₃ is W, 1-nal, 2-nal, A or F;

### X₁₄ is D, E, I, L or V;

X₁₅ is C, A, K, a-amino-γ-bromobutyric acid or homocysteine (Hoc)
where either X₆ or X₁₅ is C or Hoc.

EPO mimetic peptides show particularly good activity in cyclized form. Usually, therefore, two peptide monomers (the monomers correspond to binding domains) are in each case cyclized with an EPO mimetic consensus and bound to a dimer, as binding to the EPO receptor is the most effective in this form. The EPO mimetic monomers have on average 10 to 25 amino acids. Preferably, as described in PCT/EP2005/012075 (WO 2006/050959), they are synthesized as continuous dimers (bivalent peptides), in order to avoid separate dimerization steps.

Various methods for the formation of intramolecular disulfide bridges for cyclization of EPO mimetic or also TPO mimetic peptides are known in the prior art. The core of the known teachings is oxidation of the cysteine residues (or other corresponding amino acids containing SH groups) in the EPO mimetic consensus. DMSO has been used until now as a typical oxidizing agent, but it has the disadvantages described at the beginning.

Cyclization by the method according to the invention has some decisive advantages over the methods known in the prior art. Thus, better yields and greater product purity are achieved than with the method known in the prior art. Another decisive advantage of the method according to the invention is that the cyclization reagent according to the invention can be separated easily from the reaction product by simple HPLC. According to another variant, the heterocyclic compound (e.g. caffeine) can be removed by liquid-liquid extraction. For example, caffeine can be removed from an aqueous peptide solution by repeated extraction with dichloromethane. In the cyclization of longer peptides it is also possible to use size exclusion chromatography (SEC). Purification is therefore greatly simplified.

Depending on the substance or peptide containing SH groups and the reaction conditions used, the reaction time can be reduced to under eight hours (e.g. by lowering the pH; choice of an additional oxidizing agent). Usually the reaction time is <=twenty-four hours, preferably under twenty hours, especially preferably under fifteen hours and quite especially preferably between five and ten hours.

Apart from the EPO mimetic peptides mentioned, however, other peptides were also cyclized successfully by the method according to the invention. Thus, among others, on the peptide derived from oxytocin, which in contrast to oxytocin has a carboxylic acid at the C terminus instead of an amide:

H-CYIQNCPLG-OH

which is also cyclized by formation of an intramolecular cysteine bridge.

As mentioned, the intramolecular disulfide bridge is preferably formed between two amino acids. These can be natural or nonnatural, the only precondition is the ability to form a disulfide bridge by reaction of the SH group. Cysteine is certainly the best known disulfide bridge-forming amino acid, and is also mainly employed in nature for forming disulfide bridges. Disulfide bridges occur in nature in particular in the formation of intra- and intermolecular disulfide bridges. For example, they are responsible for holding together the individual polypeptide chains of proteins (e.g. insulin) in the form of intermolecular disulfide bridges and, within a protein, they regularly stabilize the conformation through the formation of intramolecular disulfide bridges.

The keratin in wool and in hair for example contains more than 10% cysteine, therefore many disulfide bridges are also present there. If these disulfide bridges are broken (e.g. with alkaline solutions, light, heating etc.), the breaking strength of the fibers decreases sharply. The method according to the invention can therefore also be used for forming disulfide bridges in fibers (natural and synthetic fibers). The same applies to the treatment of hair, where disulfide bridges are also very important for the structural strength. The method according to the invention can therefore also be used for forming disulfide bridges in hair, which also opens up applications in the field of cosmetics (e.g. shampoos, reagents for permanent waving etc.). Thus, the method according to the invention can be used for example as an agent for closing disulfide bridges in the area of permanent wave treatment. For this it is especially advantageous if in addition to the substance characterized more precisely above for the promotion of disulfide bridge formation, an oxidizing agent is added. It has been shown that this can greatly accelerate the reaction rate and the disulfide bridges are closed correspondingly more quickly. This has the result that when the method according to the invention is used on hair, the time of action and therefore also the treatment time are shorter, which is advantageous for the customer. An especially suitable oxidizing agent is oxidized glutathione (GSSG). The resultant improvement in closing of the disulfide bridges, in quantitative terms and in terms of time, is described concretely in the experimental examples.

Accordingly, the present invention also relates to the use of the heterocyclic compounds described above in cosmetic preparations. With these cosmetic preparations, the formation of disulfide bridges can be promoted correspondingly, for example in the case of hair.

The cosmetic preparations can contain, as well as the heterocyclic compound described previously, suitable solvents and the additives that are usual in such formulations. We may mention for example emulsifiers and coemulsifiers, surfactants, oils, preservatives, perfume oils, cosmetic care and active substances such as AHA acids, fruit acids, ceramides, phytanetriol, collagen, vitamins and pro-vitamins, for example vitamin A, E and C, retinol, bisabolol, panthenol, natural and synthetic sunscreen agents, natural substances, opacifiers, micropigments such as titanium dioxide or zinc oxide, overgreasing agents, pearly luster wax, consistency agents, thickeners, solubilizers, complexing agents, fats, waxes, silicone compounds, hydrotropes, dyes, stabilizers, pH regulators, reflectors, proteins and hydrolyzed proteins, hydrolyzed albumen, salts, gelling agents, silicones, humectants, regressing agents and other usual additives. In addition, for adjusting the properties that are desired in each particular case, polymers can be included.

For protecting the hair against damage by UV radiation, the cosmetic preparations can also contain UV sunscreen agents.

Hair-cosmetic preparations include in particular styling agents and/or conditioners in hair-cosmetic preparations such as medicated hair-care products, hair foams, hair gels, hair sprays, hair lotions, hair rinses, hair shampoos, hair emulsions, leveling agents for permanent waves, hair dyes and bleaches, setting lotions or similar products. Depending on the area of application, the hair-cosmetic preparations can be applied as (aerosol) spray, (aerosol) foam, gel, gel spray, cream, lotion, milk or wax.

Preferably the agent is a product for the hair, which is selected from shampoos and products for the hair, which are or are not rinsed out and are applied before or after hair washing, dyeing, decolorizing, permanent waving or straightening.

According to the invention, therefore, also a method is provided for the treatment of hair, which is characterized in that the hair is brought into contact with the cosmetic agent, containing at least one of the heterocyclic compounds described previously and optionally is rinsed with water. The heterocyclic compound is preferably selected from the compounds and classes of compounds discussed in detail above.

Moreover, the method as presented can also be used for forming disulfide bridges of synthetic substances, which only have corresponding functional groups bearing SH groups, but for example are not formed from amino acids (but for example from an organic polymer).

According to an advantageous further development, it is far easier to remove the substance that promotes the formation of disulfide bridges. According to this concept, a support is charged with the substance that promotes disulfide bridges. The support can be e.g. a (hydrophilic) resin. As a result of binding of the substance to the support, the supported substance can be removed e.g. by simple filtration. Therefore it may be advantageous to use caffeine or caffeine-like substances (see above formula) in the above method of formation of disulfide bridges, which are bound to a support in order to facilitate removal.

To make it possible for the substance to bind to the support, functional groups on the substance are helpful. Therefore it is also possible to use derivatives of the substance that forms the disulfide bridges.

Examples of suitable caffeine derivatives are:

theophylline-8-butyric acid

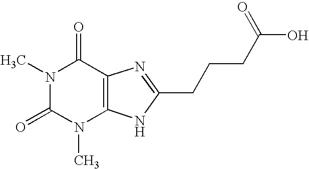

and
theophylline-7-acetic acid

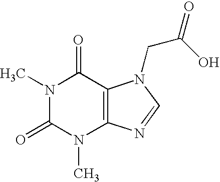

Both derivatives promote the formation of disulfide bridges and hence also the cyclization of peptides in solution. If these substances are bound covalently to a suitable support via their functional group, an immobilized reagent is obtained, which is able to accelerate the closing of disulfide bridges. After the reaction, the reagent can be removed from the reaction solution by simple filtration. As is clear on the basis of these compounds, according to the invention it is also possible to attach residues, such as here in the case of 8-(3-carboxypropyl)-1,3-dimethylxanthine for example a functional group such as R₅′ for coupling to the carrier substance in the remaining positions on the heterocyclic ring, independently of the residues R₁′ to R₃′.

The invention also relates to the use of the heterocyclic compounds described above for forming disulfide bridges, in particular intra- or intermolecular disulfide bridges in peptides and proteins.

The substances to be used according to the invention are particularly suitable for the cyclization of peptides, in particular EPO mimetic peptides, by forming intramolecular disulfide bridges. Especially preferred examples are N-methyl-2-pyridone, 2,6-dihydroxy-pyridine hydrochloride, uracil-6-carboxylic acid, 2,4-dihydroxy-6-methyl-pyrimidine, 2,4-dimethyl-6-hydroxypyrimidine, 2-isopropyl-6-methyl-4-pyrimidinol, 4,6-dihydroxy-2-methylpyrimidine, 4,6-dihydroxypyrimidine, 1,2-dihydro-3,6-pyridazinedione, 7-hydroxy-5-methyl[1.2.4]triazolo[1,5-a]pyrimidine, barbituric acid, alloxan monohydrate and violuric acid, uracil, 1-methyl-uracil, 3-methylxanthine, theobromine, theophylline, caffeine, isocaffeine, xanthine, theophylline-7-acetic acid, theophylline-8-butyric acid, 3-isobutyl-1-methylxanthine, 1,2,3-benzotriazin-4(3H)-one, (−)-riboflavin, lumazin, alloxazin, minoxidil (=6-(1-piperidinyl)-2,4-pyrimidinediamine-3-oxide) and aminexil (=2,4-diaminopyrimidine-3-oxide).

These substances can be used particularly well according to the invention for forming disulfide bridges. The following are especially preferred:

N-methyl-2-pyridone, barbituric acid, alloxan monohydrate, violuric acid, 4,6-dihydroxypyrimidine, uracil-6-carboxylic acid, minoxidil, 3-methylxanthine, theobromine, theophylline, 3-isobutyl-1-methylxanthine, caffeine, isocaffeine, lumazin, alloxazin.

The disulfide bridges are formed between SH-containing groups. In particular, natural and nonnatural amino acids having free SH groups are suitable disulfide bridge forming agents.

Based on their capacity for promoting the formation of disulfide bridges, the substances of the above formula can be used for example for the treatment of substances and materials containing SH groups, in order to promote the formation of disulfide bridges. Thus, the substances can be used e.g. for the treatment of hair or fibers (natural and synthetic fibers). This applies in particular to cysteine-containing fibers. The heterocyclic compounds that are to be used according to the invention can also be used for example in liquid formulations (e.g. in the form of rinses or shampoos or other agents for treatment of the hair, for example perming reagents). Corresponding compositions are therefore also covered by the invention.

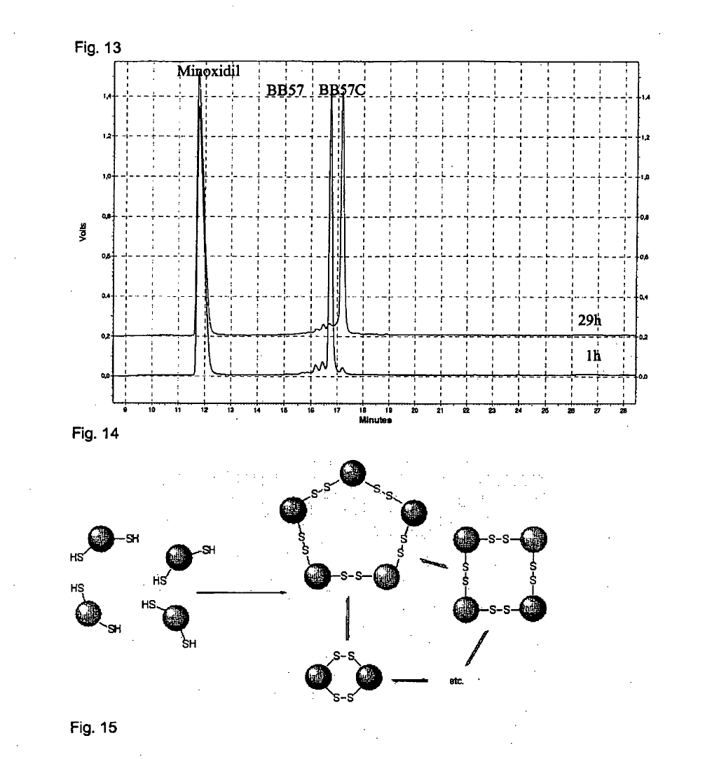

On the basis of the catalytic action of the heterocyclic compounds characterized according to the invention on the formation of disulfide bridges, they can for example also be used for catalysis in the formation of inter- or intramolecular disulfide bridges for the production of dynamic combinatorial libraries. They can therefore be used for forming disulfide bridges between synthetic or natural or modified natural molecules. They can therefore find application in the production of dynamic combinatorial libraries for searching for active substances. In the case of dynamic combinatorial libraries, the individual units are often crosslinked by means of disulfide bridges to form macromolecules (see FIG. 15). Details for the libraries are described for example in “Dynamic combinatorial libraries of macrocyclic disulfides in water. S. Otto, R. L. E. Furlan and J. K. M. Sanders, J. Amer. Chem. Soc., 2000, 122, 12063-12064”; “Selection and amplification of hosts from dynamic combinatorial libraries of macrocyclic disulfides. S. Otto, R. L. E. Furlan and J. K. M. Sanders, Science, 2002, 297, 590-593”; “Drug discovery by dynamic combinatorial libraries. Ramström, Lehn. Nat. Rev Drug Discov. 2002” and WO01/64605.

The method according to the invention will now be explained with some examples. EPO mimetic peptides and oxytocin were chosen as examples of peptides that can be cyclized by the method according to the invention.

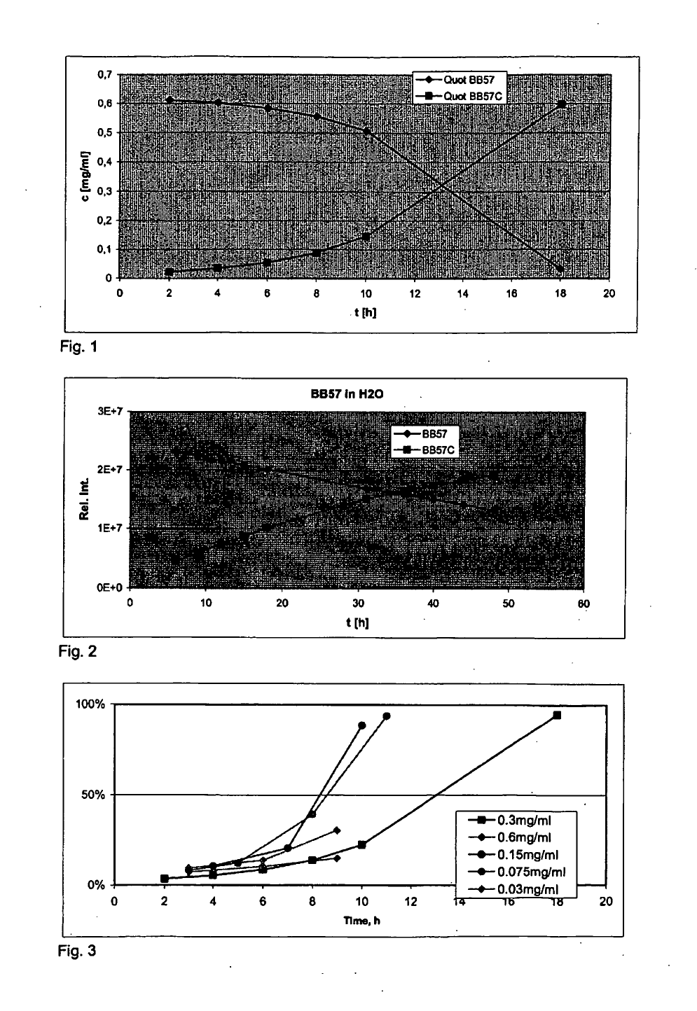

FIG. 1 shows the course of the reaction of cyclization of an EPO mimetic peptide of the following sequence

GGTYSCHFGKLTWVCKKQGG-Am (BB570)

(0.7 mg/ml) to the corresponding cyclized product in the presence of caffeine (0.3 mg/ml) with air in water at room temperature. As the curve clearly shows, the reaction rate increases in the course of the reaction, which suggests an autocatalytic mechanism of the reaction. The abbreviation Am generally stands for an amidation.

FIG. 2 shows the cyclization of the same peptide as in FIG. 1 (0.7 mg/ml) to its cyclized form in the absence of caffeine. It can clearly be seen that the reaction rate has decreased considerably.

In the case of EPO mimetic peptides, the optimal concentration of caffeine was found to be in a range from 0.075 to 0.15 mg/ml. Conversion is already quantitative after ten hours.

FIG. 3 shows the rate of conversion of the EPO mimetic peptide shown in FIG. 1 as a function of the caffeine concentration. As can be seen, very good results can be achieved in a concentration range from 0.03 mg/ml to 0.3 mg/ml. The optimal values are in a range from 0.06 mg/ml or 0.075 to 0.15 mg/ml.

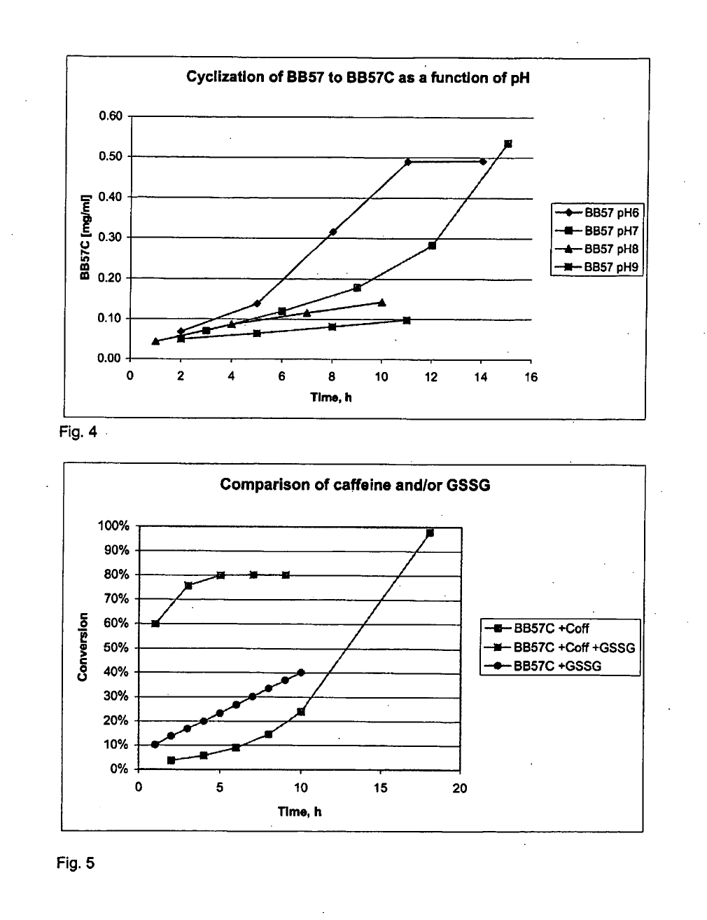

FIG. 4 shows the rate of cyclization as a function of the pH value. As can clearly be seen, the autocatalytic course of the reaction cannot be attributed to a change in pH value, as this effect also occurs in the buffered solutions shown (phosphate buffer, pH 6 to 9). However, it was found that the yield of cyclized peptide decreases at higher pH values. Moreover, it was found, surprisingly, that the reaction goes more quickly, the lower the pH value of the solution. Therefore a lower pH value of below 7 and preferably <=6.5 is preferred.

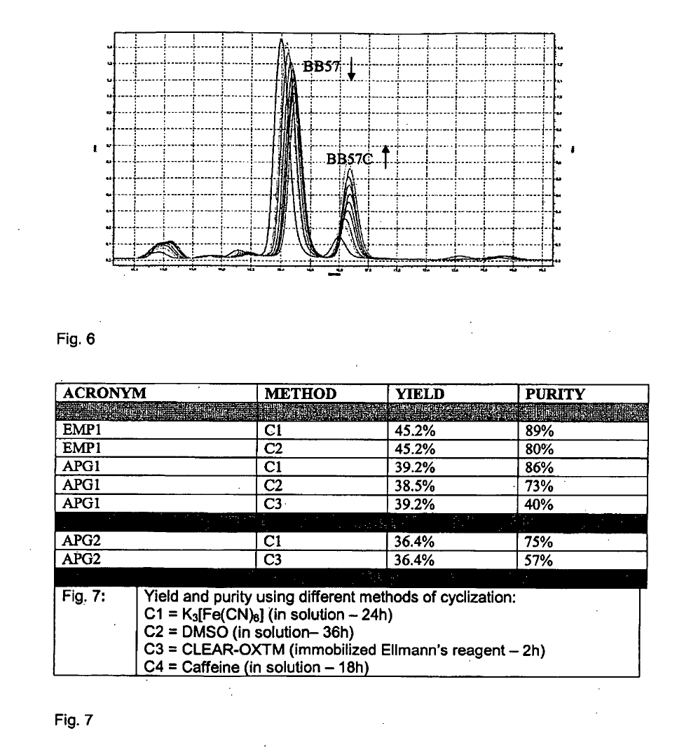

FIG. 5 shows the influence of the mild oxidizing agent glutathione (oxidized form) on the reaction rate. Here, conversion of the peptide shown in FIG. 1 (0.7 mg/ml H₂O) took place in the presence of 0.1 mg/ml (0.5 equivalent) glutathione, oxidized form (GSSG) and caffeine (0.3 mg/ml). The reaction was already completed within five to six hours. Conversion of the peptide only takes place slowly with GSSG alone (in the absence of caffeine). It was also found that undesirable by-products are formed (see FIG. 6).

FIG. 6 shows chromatograms, recorded in each case after reaction for one hour, which provide evidence of conversion of the EPO mimetic peptide used with 0.5 equiv. GSSG.

FIG. 7 shows a synoptic table comparing the method according to the invention with the methods known in the prior art. The peptides tested had the following sequences:

EMP1: Ac-GGTYSCHFGPLTWVCKPQGG-Am
APG1: Ac-GGTYSCHFGKLTWVCKKQGG-Am
APG2: Ac-GGTYSCHFGKLT-Na1-VCKKQRG-Am

The in vitro experiments showed comparable activity of the peptides cyclized by the various methods. The method according to the invention is characterized, however, by better yields and purities relative to the other cyclization methods tested, as clearly demonstrated in FIG. 7. A further advantage of the method according to the invention is that the cyclization reagent used can be removed easily by HPLC.

Further examples of EPO mimetic peptides cyclized by the method according to the invention are shown below:

Ac-C(tBu)-GGTYSCHFGKLT-Nal1-VCKKQRG-GGTYSCHFGKLT-
Nal1-VCKKQRG-Am (APG3)
Ac-C(Mob)-GGTYSCHFGKLT-Nal1-VCKKQRG-GGTYSCHFGKLT-
Nal1-VCKKQRG-Am (APG4)
Ac-C(tBu)-GGTYSCHFGKLTWVCKKQGG-GGTYSCHFGKLTWVCKKQG
G-Am (APG5)
(Sama)-GGTYSCHFGKLT-Na1-VCKKQRG-GGTYSCHFGKLT-Na1-V
CKKQRG-Am (APG6)

FIG. 8 shows the cyclization of dimeric EPO mimetic peptides. The cyclization of di- or multimeric peptides preferably takes place in several steps. FIG. 8 shows the synthetic scheme based on a bivalent (dimeric) EPO mimetic peptide, which is cyclized in 2 steps by formation of two intramolecular disulfide bridges. According to this method, the first disulfide bridge is formed by the method according to the invention. The second intramolecular disulfide bridge was formed by carrying out an optimized iodine oxidation. For coupling the peptide to a polymeric carrier, some additional cysteine residues were inserted in the molecule. This cysteine was protected with suitable protective groups (tBu or Mob).

The first cyclization according to the invention using caffeine is preferably carried out at pH 6, whereas the second cyclization, according to the example shown, took place in 80% acetic acid. The synthesis yield was typically between 60 and 90%.

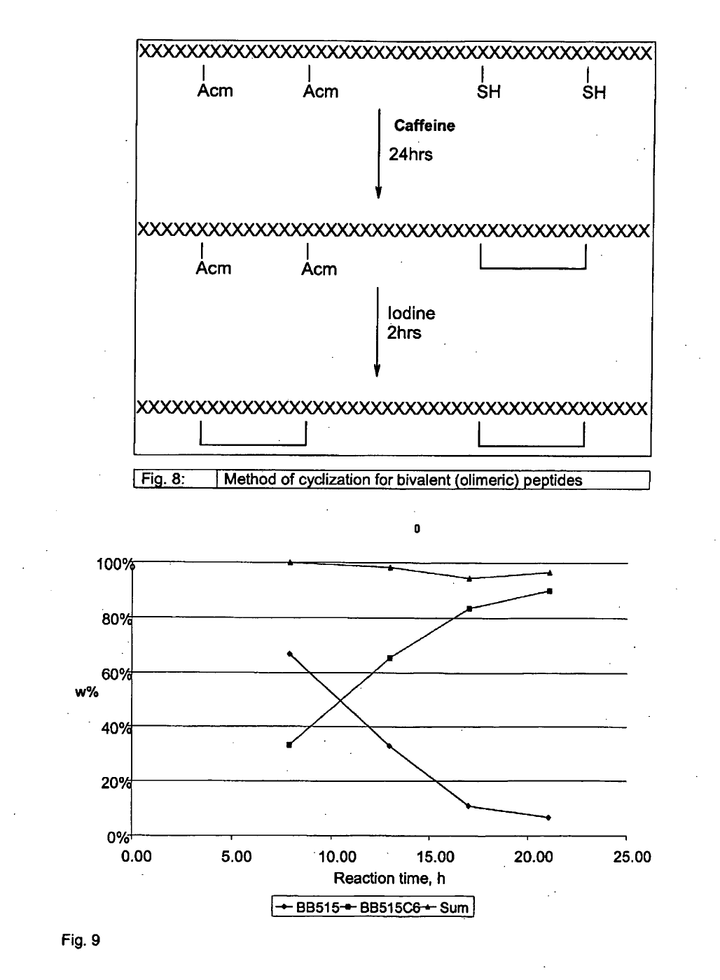

FIG. 9 Some of the EPO mimetic peptides can only be cyclized with great difficulty. An example is the following peptide:

Ac-GGTYSCHFG-Har-LT-1-Nal-VCK-Aad-Q-Aad-G-NH₂
i. +-----------------+

### Har=Homoarginine

Aad=2-aminoadipic acid, “homoglutamic acid”
Nal: naphthylalanine

With this sequence it was found to be advantageous to increase the concentration of cyclization reagent. Thus, cyclization with 10 mg/ml caffeine was successful within 24 h. The results are shown in FIG. 9. The yield after approx. 21 h in solution was already >90%.

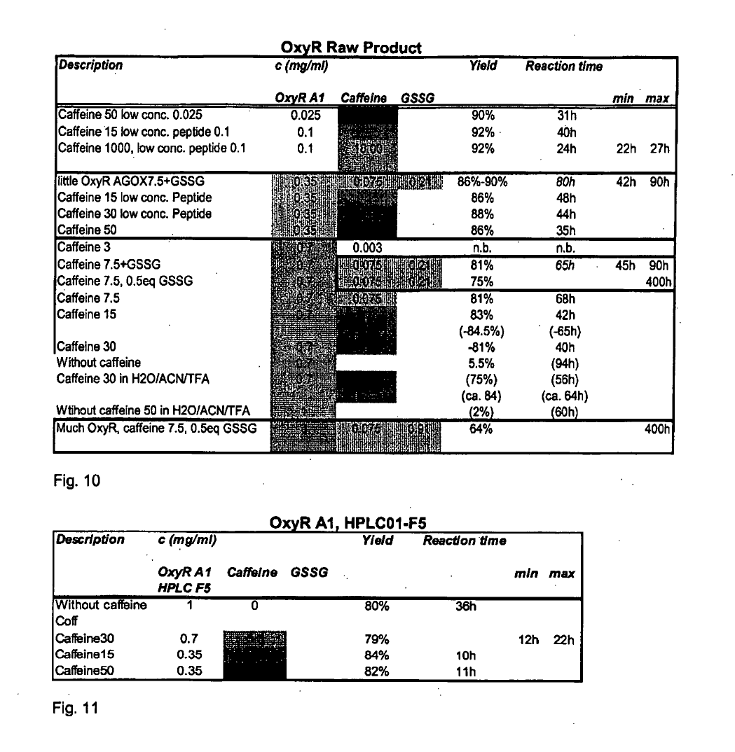

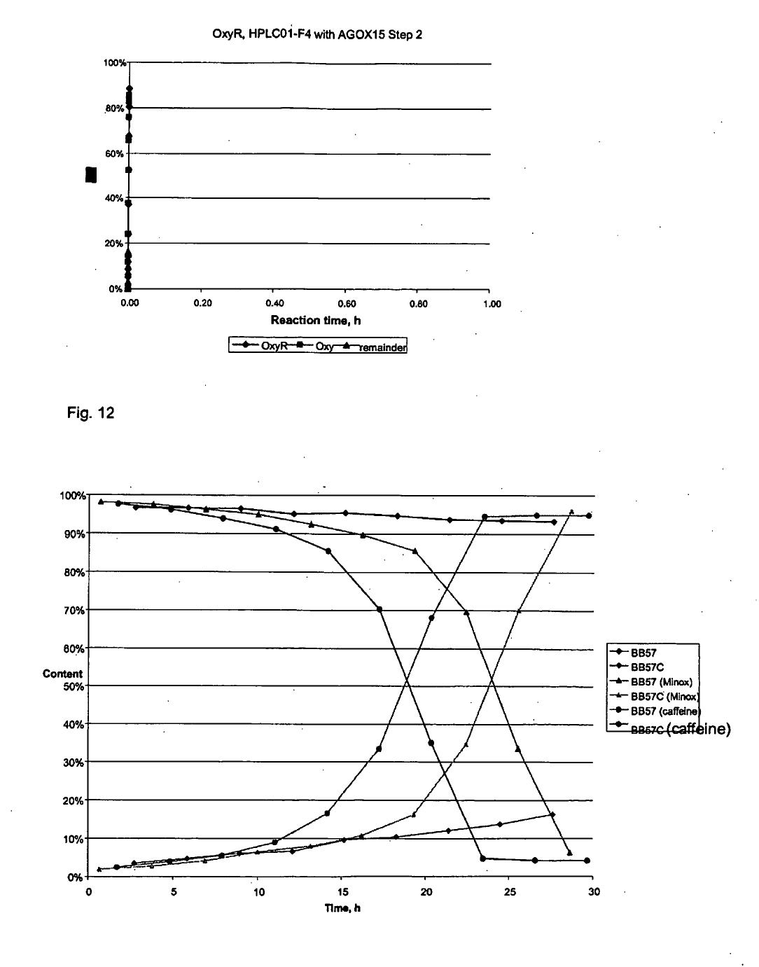

FIGS. 10 to 12 In addition to EPO mimetic peptides, a reduced peptide derived from oxytocin was also cyclized with caffeine or the caffeine-like substance (see above formula).

For carrying out the reaction, oxytocin, reduced (OxyR), raw product, was dissolved in water (or H₂O/ACN/TFA) and was left to stand in the air with various concentrations of caffeine (and optionally GSSG). The reaction mixture was analyzed by HPLC at regular intervals, in order to determine the contents of OxyR and the product oxytocin (Oxy).

The tables shown in FIGS. 10 to 12 and the corresponding graph provide a synopsis of the results.

It can be seen that product yield is correlated with the peptide concentration. In the case of oxytocin, smaller amounts of peptide lead to better results.

The reaction time up to complete conversion of OxyR correlates with the concentration of caffeine in the reaction solution. Up to a concentration of 0.5 mg/ml caffeine, the more caffeine, the faster the oxidation. The peptide concentration only has a minor influence on the reaction time.

GSSG seems to have no influence on rate or yield.

OxyR, HPLC-purified, already cyclizes spontaneously “really” quickly. The reaction rate can, however, still be shortened considerably with caffeine. Small amounts of ACN/TFA have a slight influence on yield, and the reaction time is somewhat longer.

FIGS. 13 and 14 show the results of cyclization of peptide BB57 with the substance minoxidil:

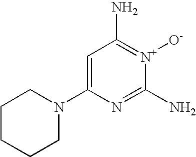

For this, 0.7 mg BB57 and 0.3 mg minoxidil (6-(1-piperidinyl)-2,4-pyrimidinediamine-3-oxide, Minox) were dissolved in 1 ml distilled water and left to stand in the air. The conversion of BB57 to oxidized BB57C was monitored by HPLC (UV detection at 216 nm). The results are shown in FIG. 13.

As shown in FIG. 13, oxidation in the presence of minoxidil is completed after approx. 29 h (in a comparative measurement with caffeine the figure is approx. 24 h). Minoxidil, as another representative of the heterocyclic compounds according to the invention, therefore also has a positive effect on the formation of disulfide bridges. The yield in solution of the minoxidil-catalyzed reaction is above 95%. That the reaction is a catalytic reaction can be seen from the fact that the concentration of minoxidil barely decreases in the reaction (decrease=2% at 4.4 eq minoxidil relative to BB57, see FIG. 14).

FIG. 15 shows possible linking strategies with disulfide bridges for the production of crosslinked macromolecules. Such building blocks often find application in dynamic combinatorial libraries.

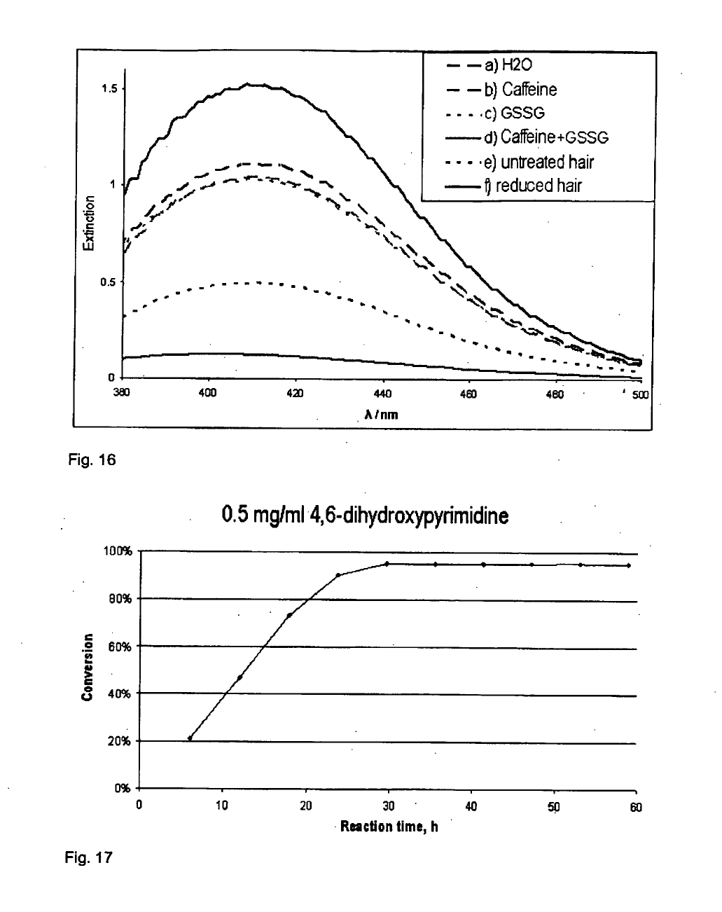

FIG. 16 In addition, tests were conducted to demonstrate that hair, previously reduced in the sense of perming, closes oxidatively at a faster rate with a combination of the substance according to the invention (in this case caffeine) and an additional oxidizing agent (in this case oxidized glutathione—GSSG) in the presence of air. Therefore the method according to the invention can also be used advantageously in the cosmetic field and in particular in hairdressing for the treatment of hair.

The test was carried out as follows:

In each case 5-6 mg of hair is treated with 400 μl of a 10% solution of a “Wave-Lotion” containing ammonium thioglycolate (product “Poly Lock—strong permanent wave”, Schwarzkopf & Henkel, Germany) for 0.5 h at room temperature. The solution is removed and the hair is then washed 6 times with 400 μl H₂O each time. One of the hair samples treated in this way is then treated in each case a) in H₂O, or an aqueous solution of b) 10 mg/ml caffeine, c) 5 mg/ml GSSG in H₂O and d) 10 mg/ml caffeine and 5 mg/ml GSSG at room temperature for 3 days.

For determination of the free thiol groups still remaining, after removing the reaction solution the hair is reacted with Eliman's reagent (5,5′-dithiobis(2-nitrobenzoic acid), DTNB). Untreated hair and reduced hair, which have not otherwise undergone further treatment, serve as additional reference samples. The hair samples are put in 200 μl each of 100 mM phosphate buffer, pH 8.0 and 1 mM EDTA, and 300 μl of a 1 mM DTNB solution in the same EDTA-containing buffer. The solution is analyzed after a few minutes in a UV-Vis spectrometer.

The UV-Vis spectra of the samples show that reduction of the hair by treatment with the ammonium thiolglycolate-containing “Wave-Lotion” was successful (see FIG. 29, curves e and f, showing on the one hand untreated hair and on the other hand reduced hair). This can be seen from the fact that the band at 412 nm—a measure of the number of free thiol groups—is largest. Treatment in the presence of air with a) H₂O, b), with a caffeine-containing solution, c) the GSSG-containing solution leads in each case only to partial oxidation of the thiol groups. However, it can already be seen that caffeine has a beneficial influence on closing of the disulfide bridges. The combination d) of caffeine and the oxidizing agent GSSG led, however, to almost quantitative oxidation of the thiol groups in the case of hair. This test therefore demonstrates that the method according to the invention for closing disulfide bridges can also be used successfully on hair.

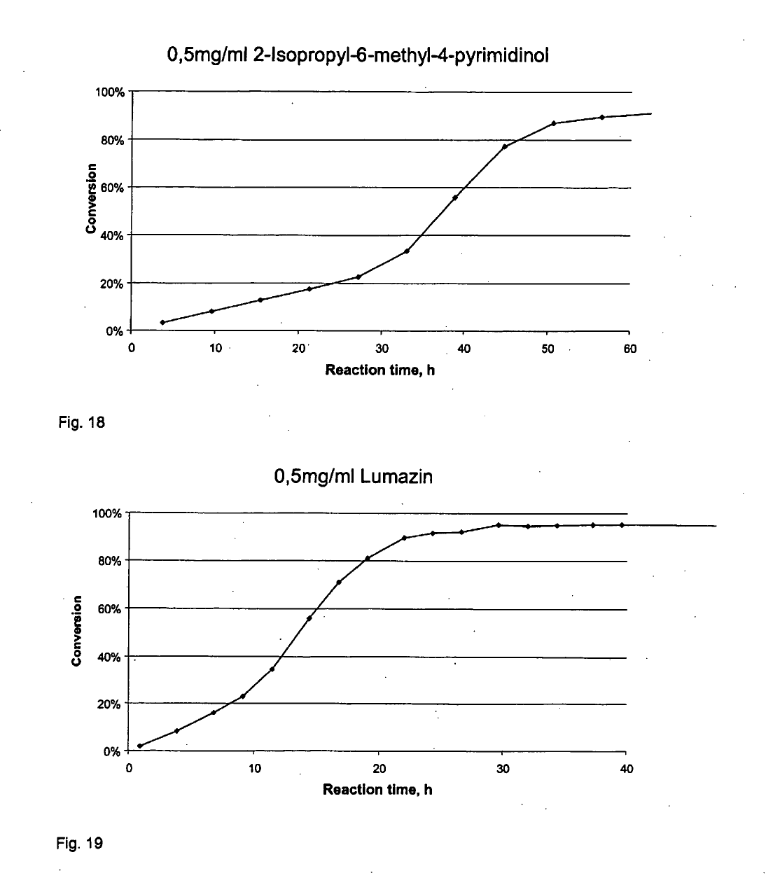

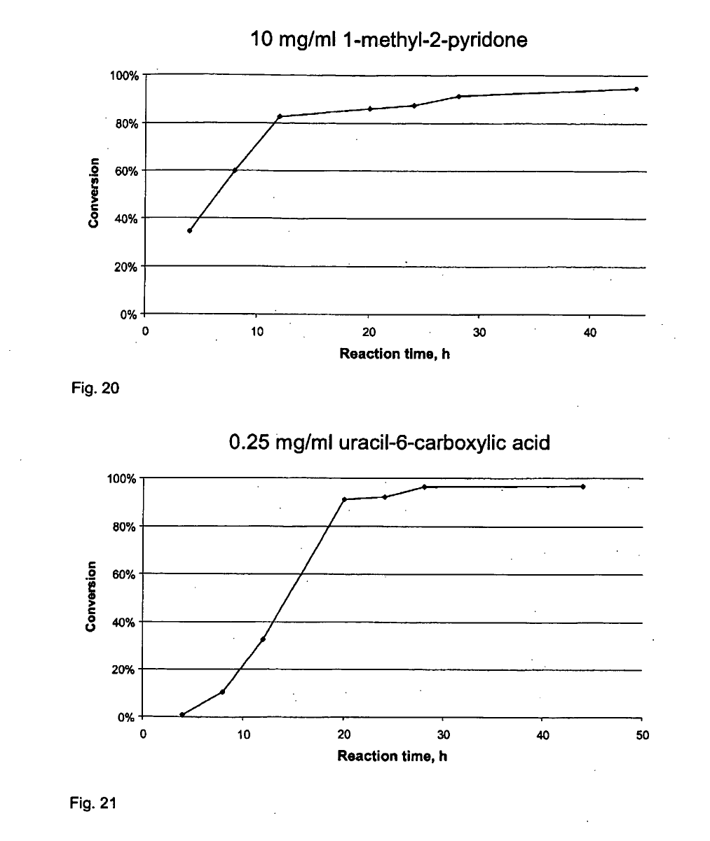

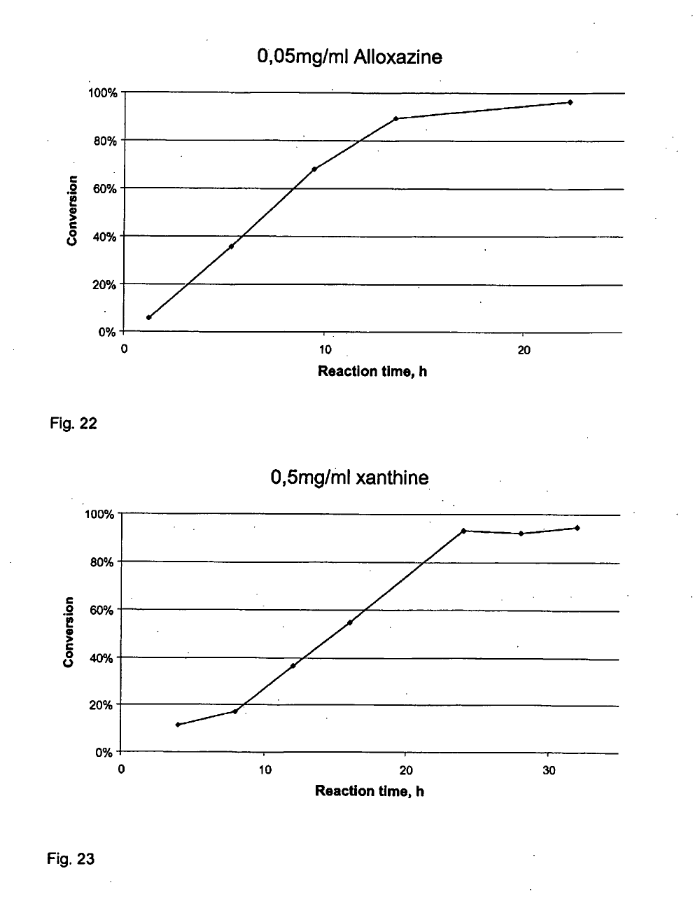

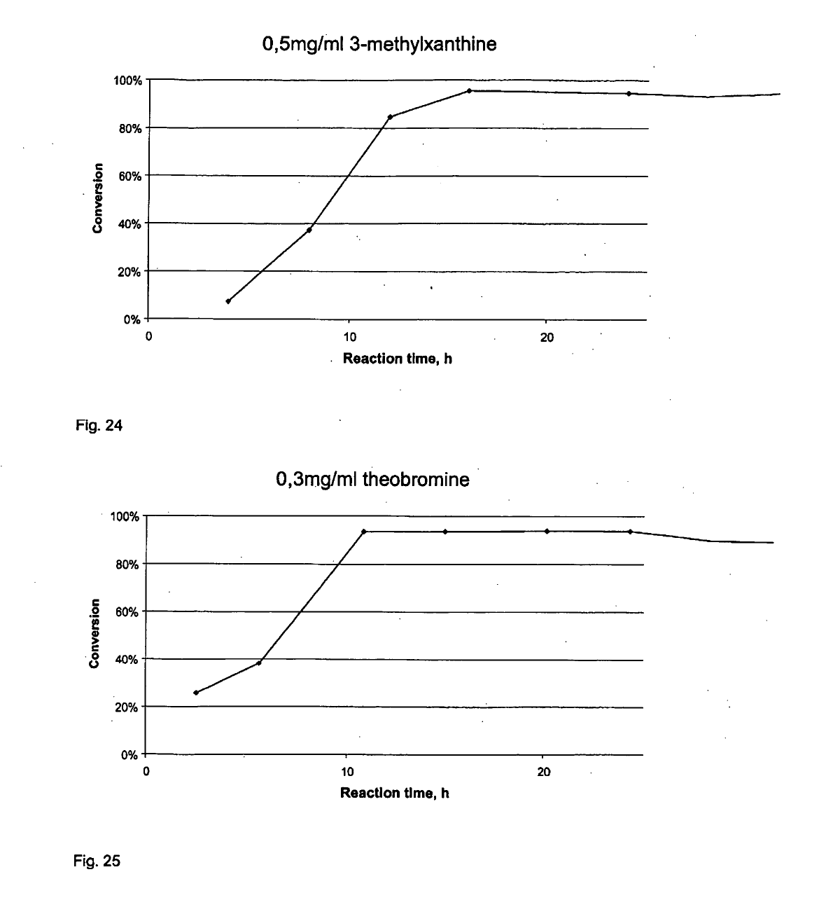

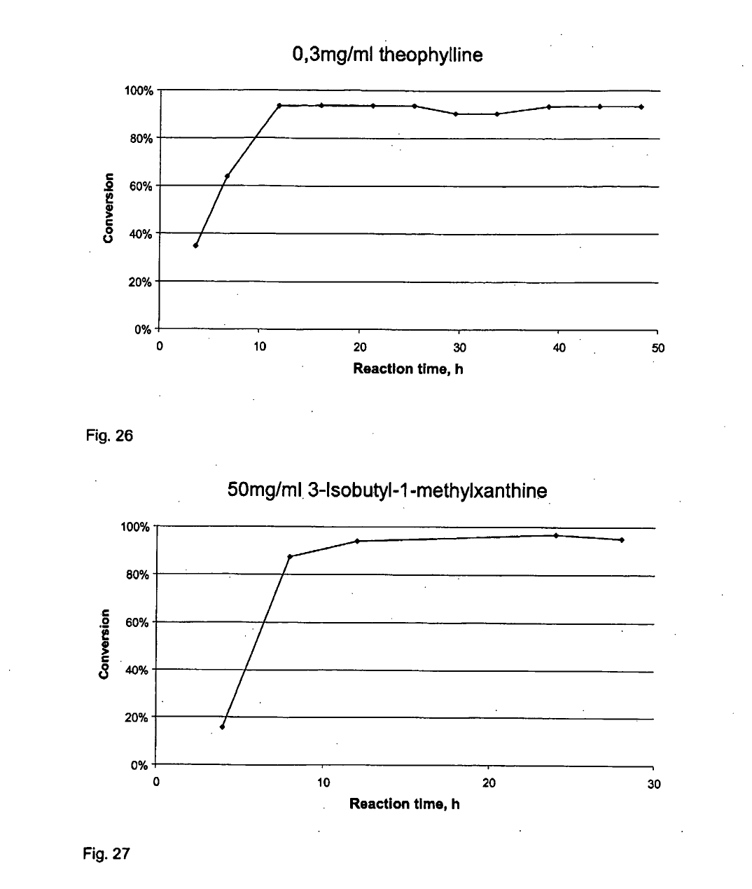

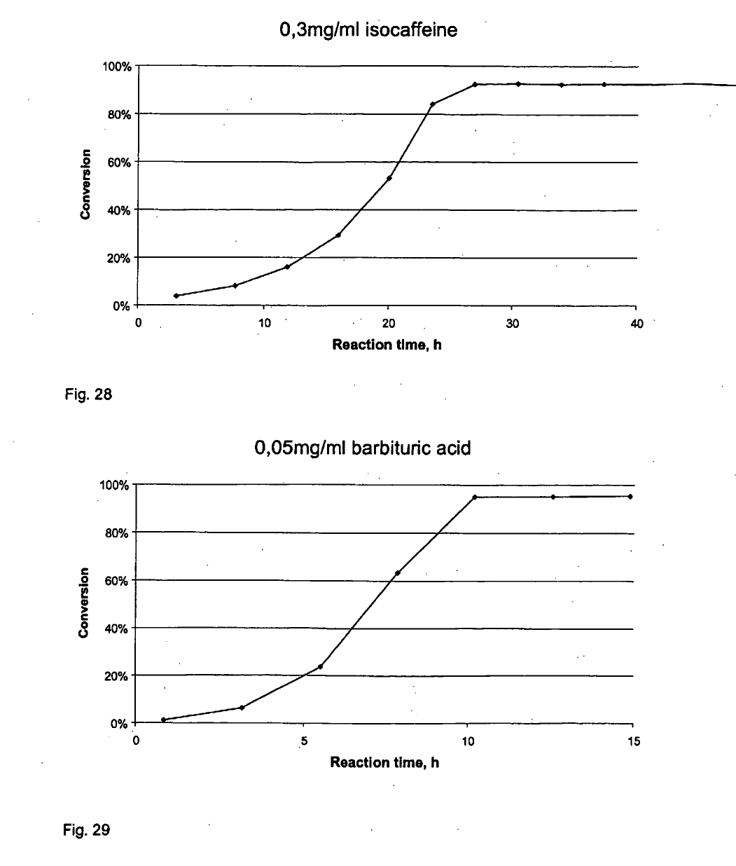

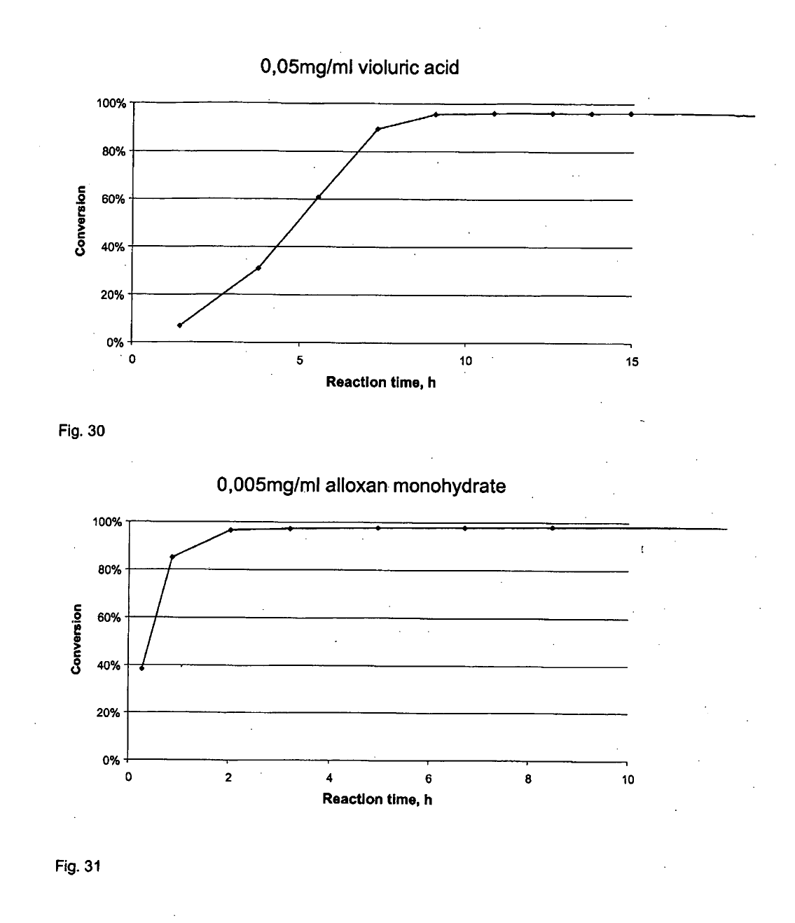

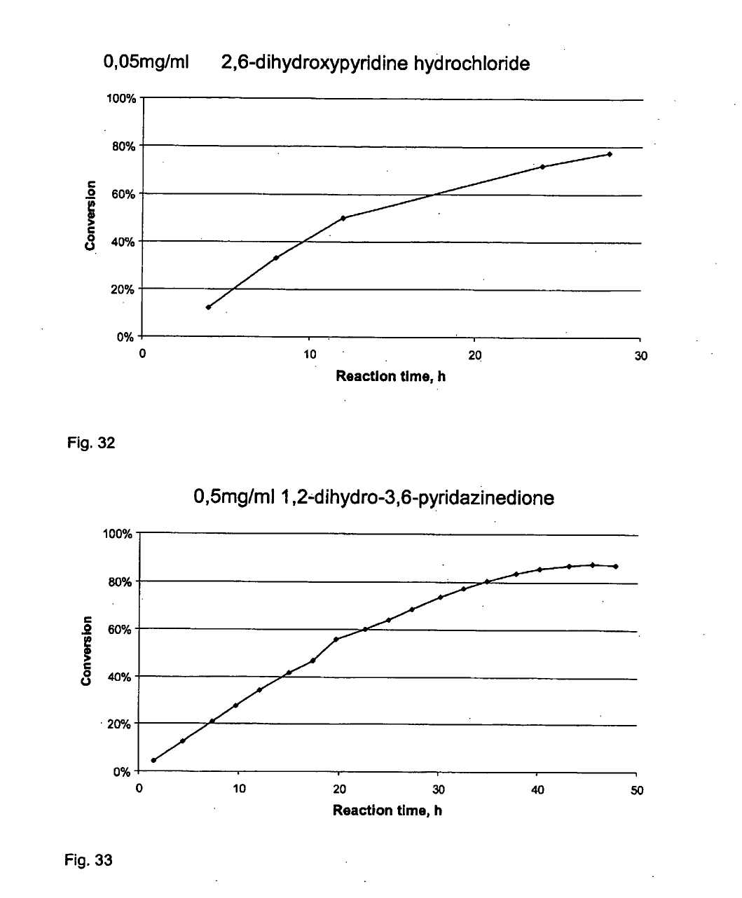

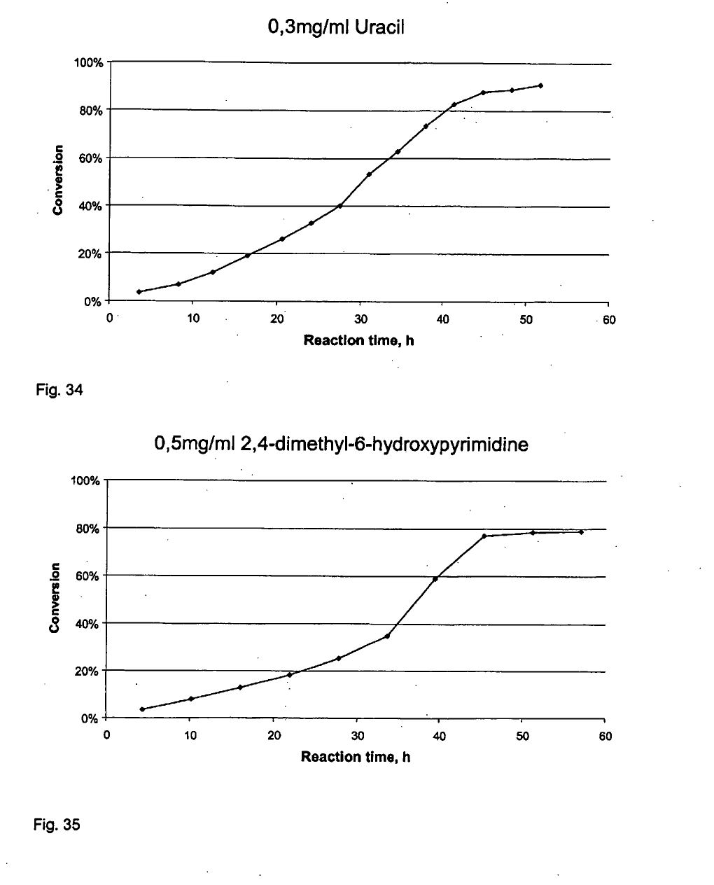

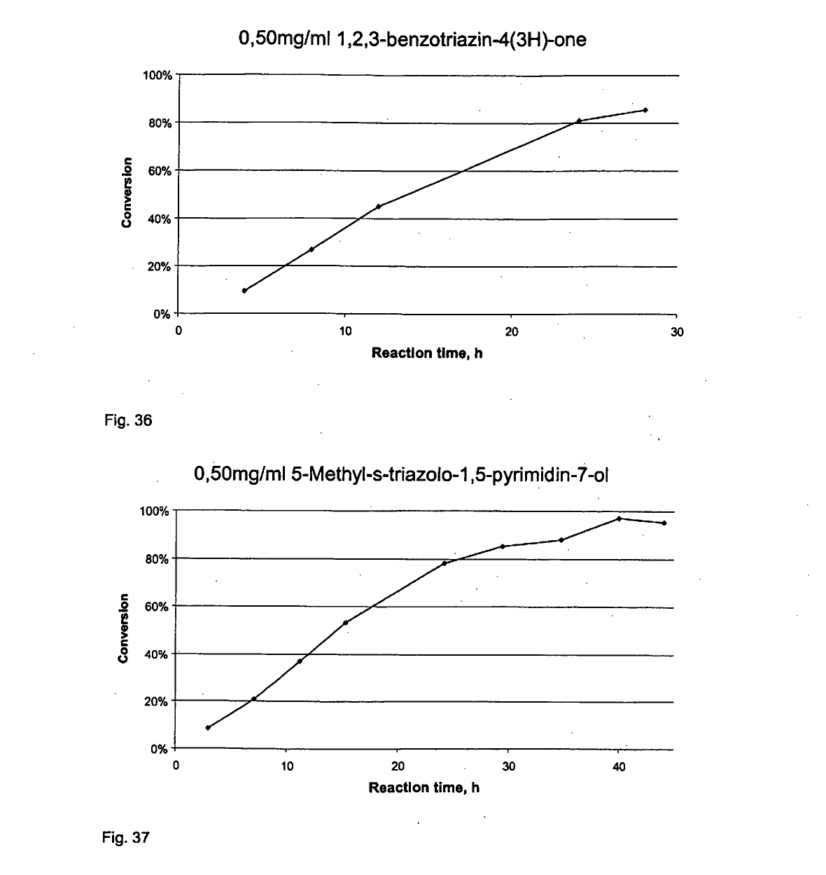

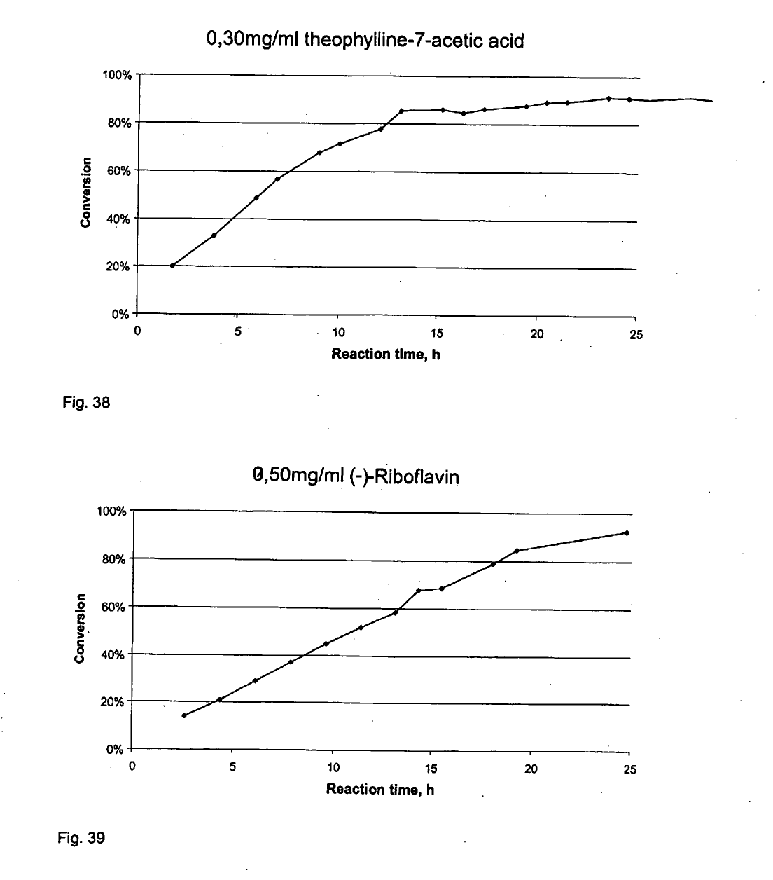

FIG. 17 to 39 show the results of cyclization of the reference peptide BB57 with various substances which can, according to the invention, promote the formation of disulfide bridges.

The tests were carried out as described previously (see above). The substance tested and the amounts tested are stated in each case.

1. A method of formation of disulfide bridges, characterized in that the reaction is carried out in a liquid medium that contains at least one compound which promotes the formation of disulfide bridges, said compound being selected from the following group:
(a) a compound having in its structure a saturated or unsaturated six-membered heterocycle with at least one nitrogen atom, said heterocycle having at least one hydroxyl group or an oxo group (═O) according to the invention on the carbon atom adjacent to the nitrogen atom, and if a hydroxyl group is present the heterocycle is unsaturated;
(b) a compound of the following general formula 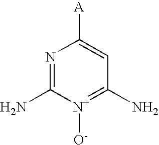
in which substituent A stands for
hydrogen, an optionally substituted alkyl residue, an optionally substituted aryl residue or a saturated or unsaturated heterocyclyl with 3 to 10 ring members and 1 to 3 heteroatoms, such as nitrogen, oxygen and/or sulfur, the heterocyclyl being unsubstituted or substituted one or more times with halogen, alkyl with 1 to 4 carbon atoms, cyano, nitro, cycloalkyl with 3 to 6 carbon atoms, hydroxy, alkoxy with 1 to 4 carbon atoms and/or mercapto.

2. The method as claimed in claim 1, characterized in that the compound according to alternative (a) has the following basic structure 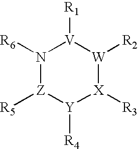
where the heterocycle is saturated or unsaturated, depending on the choice of the substituents R1 to R6, and accordingly can have one or more double bonds;
V, W, X, Y and Z represent either carbon atoms or nitrogen atoms, the heterocycle having in total not more than three nitrogen atoms;
R1 represents either a hydroxyl group or an oxo group (═O) according to the invention;
R2 and R3, always independently of one another, stand for hydrogen, an optionally substituted alkyl or aryl residue, an optionally substituted residue —(CH₂)ₙCOOX with n equal to 0 to 10 and X equal to hydrogen or alkyl, an electron-withdrawing substituent, a functional group such as in particular a hydroxyl group, an oxo group (═O) according to the invention, —CONH₂ or an oxime (═N—OH) or R2 together with R3 represents a five- or six-membered ring, which can also have heteroatoms and optionally carries other substituents, or R2 and/or R3 are absent;
R4 and R5, always independently of one another, stand for hydrogen, an optionally substituted alkyl or aryl residue, an electron-withdrawing substituent, a functional group such as in particular a hydroxyl group, an oxo group (═O) according to the invention, a carboxyl group, —CONH₂ or an oxime (═N—OH) or R4 together with R5 represents a five- or six-membered ring, which can also have heteroatoms and optionally carries other substituents, R5 together with R6 represents a five- or six-membered ring, which can also have heteroatoms and optionally carries other substituents, or R4 and/or R5 are absent;
R6 stands for hydrogen or an optionally substituted alkyl or aryl residue, or R6 together with R5 represents a five- or six-membered ring, which can also have heteroatoms and optionally carries other substituents, or R6 is absent.

3. The method as claimed in claim 2, characterized in that the compound contains the following substructure 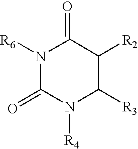
where the heterocycle is saturated or unsaturated, depending on the choice of the substituents R2, R3, R4 and R6, and accordingly can have one or more double bonds; where
R2 and R3, always independently of one another, stand for hydrogen, an optionally substituted alkyl or aryl residue, an optionally substituted residue —(CH₂)ₙCOOX with n equal to 0 to 10 and X equal to hydrogen or alkyl, an electron-withdrawing substituent, a functional group such as in particular a hydroxyl group, an oxo group (═O) according to the invention, —CONH₂ or an oxime (═N—OH) or R2 together with R3 represents a five- or six-membered ring, which can also have heteroatoms and optionally carries other substituents, or R2 and/or R3 are absent;
R4 stands for hydrogen or an optionally substituted alkyl or aryl residue or R4 is absent;
R6 stands for hydrogen or an optionally substituted alkyl or aryl residue or R6 is absent.

4. The method as claimed in claim 3, characterized in that the compound represents a uracil derivative of the following general formula: 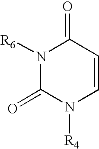
where R4 and R6, independently of one another, stand for hydrogen or an optionally substituted alkyl or aryl residue, and preferably stand for hydrogen or a linear or branched C1 to C10 alkyl residue, especially preferably for a C1 to C4 alkyl residue or hydrogen.

5. The method as claimed in claim 3, characterized in that the compound comprises purine derivatives, whose basic structure corresponds to the following general formula 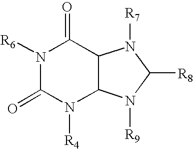
in which the five-membered ring is unsaturated and has corresponding double bonds and in which the substituents
R4 and R6, always independently of one another, stand for hydrogen, an optionally substituted alkyl residue or an optionally substituted aryl residue; preferably hydrogen, an optionally substituted C₁ to C₁₀ alkyl residue or an optionally substituted C₆ or C₁₀ aryl residue; especially preferably hydrogen, an optionally substituted C₁ to C₆ alkyl residue or an optionally substituted C₆ aryl residue; in particular for hydrogen or an optionally substituted C₁ to C₃ alkyl residue;
R7, R8 and R9, always independently of one another, stand for hydrogen, an optionally substituted alkyl residue, an optionally substituted aryl residue or an optionally substituted residue —(CH₂)ₙCOOX with n equal to 0 to 10 and X equal to hydrogen or alkyl or a functional group; preferably hydrogen, an optionally substituted C₁ to C₁₀ alkyl residue, an optionally substituted C₆ or C₁₀ aryl residue or an optionally substituted residue —(CH₂)ₙ—COOX with n=1 to 10 and X equal to hydrogen or C₁ to C₈ alkyl; especially preferably hydrogen, an optionally substituted C₁ to C₆ alkyl residue, an optionally substituted C₆ aryl residue or an optionally substituted residue —(CH₂)ₙ—COOX with n=1 to 6 and X equal to hydrogen or C₁ to C₆ alkyl; in particular hydrogen, an optionally substituted C₁ to C₃ alkyl residue or an optionally substituted residue —(CH₂)ₙ—COOX with n=1 to 4 and X equal to hydrogen or C₁ to C₃ alkyl.

6. The method as claimed in claim 1, characterized in that R2 and R3 together form a six-membered ring, which optionally has at least one heteroatom.

7. The method as claimed in claim 6, characterized in that the compound has the following basic structure: 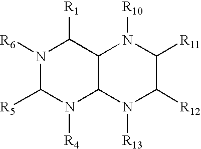
where, depending on the choice of the substituents, the rings are unsaturated and correspondingly can have one or more double bonds,
R1 represents either a hydroxyl group or an oxo group (═O) according to the invention;
R4 and R6, always independently of one another, stand for hydrogen, an optionally substituted alkyl residue or an optionally substituted aryl residue, or are absent; and preferably stand for hydrogen, an optionally substituted linear or branched C₁ to C₁₀ alkyl residue or an optionally substituted C₆ or C₁₀ aryl residue; especially preferably for hydrogen, an optionally substituted C₁ to C₆ alkyl residue or an optionally substituted C₆ aryl residue; in particular hydrogen or an optionally substituted C₁ to C₃ alkyl residue;
R5 stands for hydrogen, an optionally substituted alkyl or aryl residue, an electron-withdrawing substituent, a functional group such as in particular a hydroxyl group, an oxo group (═O) according to the invention, a carboxyl group, —CONH₂ or an oxime (═N—OH), or R5 is absent;
R10 and R13, always independently of one another, stand for hydrogen, an optionally substituted alkyl residue or an optionally substituted aryl residue, or R10 and/or R13 are absent; and preferably stand for hydrogen, an optionally substituted linear or branched C₁ to C₁₀ alkyl residue or an optionally substituted C₆ or C₁₀ aryl residue; especially preferably for hydrogen, an optionally substituted C₁ to C₆ alkyl residue or an optionally substituted C₆ aryl residue; in particular for hydrogen or a C₁ to C₆ alkyl residue substituted with at least one hydroxyl group;
R11 and R12, always independently of one another, stand for hydrogen, an optionally substituted alkyl or aryl residue, an electron-withdrawing substituent, a functional group such as a hydroxyl group, an oxo group (═O) according to the invention, a carboxyl group, —CONH₂ or an oxime (═N—OH), or R11 and R12 together form a five or six-membered ring, which optionally can have further heteroatoms and substituents.

8. The method as claimed in claim 1, characterized in that the compound according to alternative (b) has a substituent A, which stands
for hydrogen, an optionally substituted C₁ to C₁₀ alkyl residue, an optionally substituted C₆ or C₁₀ aryl residue or a saturated or unsaturated heterocyclyl with 3 to 10 ring members and 1 heteroatom, such as nitrogen, oxygen and/or sulfur, the heterocyclyl being unsubstituted or substituted one or more times with halogen, alkyl with 1 to 4 carbon atoms, cyano, nitro, cycloalkyl with 3 to 6 carbon atoms, hydroxy, alkoxy with 1 to 4 carbon atoms and/or mercapto;
preferably for hydrogen, an optionally substituted C₁ to C₆ alkyl residue, an optionally substituted C₆ aryl residue or saturated heterocyclyl with 5 or 6 ring members and 1 heteroatom, such as nitrogen, oxygen and/or sulfur, the heterocyclyl being unsubstituted or substituted one or more times with halogen, alkyl with 1 to 4 carbon atoms, cyano, nitro, cycloalkyl with 3 to 6 carbon atoms, hydroxy, alkoxy with 1 to 4 carbon atoms and/or mercapto;
in particular for hydrogen, an optionally substituted C₁ to C₃ alkyl residue or saturated heterocyclyl with 5 or 6 ring members and 1 heteroatom, such as nitrogen, oxygen and/or sulfur, the heterocyclyl being unsubstituted.

9. The method as claimed in claim 1, characterized in that the compound for promoting the formation of disulfide bridges has the following structure 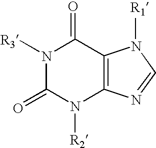
where R₁′, R₂′, R₃′ are identical or different and at least one of the residues R₁′, R₂′, R₃′ is an alkyl group.

10. The method as claimed in claim 9, characterized in that R₁′, R₂′, R₃′ are identical or different and represent either hydrogen or a C₁ to C₅ alkyl group, in particular a C₁ to C₃ alkyl group, especially preferably a methyl group.

11. The method as claimed in claim 10, characterized in that the compound is selected from the group comprising N-methyl-2-pyridone, 2,6-dihydroxy-pyridine hydrochloride, uracil-6-carboxylic acid, 2,4-dihydroxy-6-methylpyrimidine, 2,4-dimethyl-6-hydroxypyrimidine, 2-isopropyl-6-methyl-4-pyrimidinol, 4,6-dihydroxy-2-methylpyrimidine, 4,6-dihydroxypyrimidine, 1,2-dihydro-3,6-pyridazinedione, 7-hydroxy-5-methyl[1.2.4]triazolo[1,5-a]pyrimidine, barbituric acid, alloxan monohydrate and violuric acid, uracil, 1-methyl-uracil, 3-methylxanthine, theobromine, theophylline, caffeine, isocaffeine, xanthine, theophylline-7-acetic acid, theophylline-8-butyric acid, 3-isobutyl-1-methylxanthine, 1,2,3-benzotriazin-4(3H)-one, (−)-riboflavin, lumazin, alloxazin, minoxidil and aminexili.

12. The method as claimed in claim 11, characterized in that at least one intramolecular disulfide bridge is formed in amino acid-containing substances, in particular in peptides and proteins, the reaction being carried out in an aqueous medium.

13. The method as claimed in claim 12, characterized in that an intramolecular disulfide bridge is formed between two amino acids, which have an SH group, preferably between two cysteine residues.

14. The method as claimed in claim 13, characterized in that an oxidizing agent, preferably glutathione in oxidized form, is added to the reaction mixture.

15. The method as claimed in claim 14, characterized in that the peptides have a length between 5 and 100, 5 and 50 amino acids, preferably between 10 and 40, especially preferably between 15 and 25 amino acids.

16. The method as claimed in claim 15, characterized in that the substance is bound to a support.

17. The method as claimed in claim 16, characterized in that the substance is bound to the support via functional groups.

18. Use of a heterocyclic compound as claimed in claim 1 for forming disulfide bridges.

19. The use as claimed in claim 18, characterized in that the heterocyclic compound is used for the cyclization of peptides and/or proteins, the peptides preferably having a length between 5 and 250, 5 and 100, 5 and 50, preferably 10 to 40, especially preferably between 15 and 25 amino acids.

20. The use as claimed in claim 18, characterized in that the substance is used for the cyclization of cytokine-mimetic peptides, in particular EPO mimetic or TPO mimetic peptides.

21. The use as claimed in claim 18 for formation of an intramolecular disulfide bridge between at least two amino acids bearing SH groups.

22. The use as claimed in claim 18 for the treatment of substances bearing SH groups and products for forming disulfide bridges.

23. The use as claimed in claim 22 for the treatment of hair and fibers, in particular cysteine-containing fibers.

24. A composition containing a heterocyclic compound as claimed in claim 1 for forming and/or promoting disulfide bridges.

25. Use of the composition as claimed in claim 24 for the treatment of hair and/or fibers.

26. Use of a heterocyclic compound as claimed in claim 1 for catalysis in the formation of inter- or intramolecular disulfide bridges for the production of dynamic combinatorial libraries.

27. A cosmetic agent for promoting the formation of disulfide bridges, characterized in that it contains a heterocyclic compound that has at least two nitrogen atoms, as claimed in claim 1.

28. A method for the treatment of hair, characterized in that the hair is brought into contact with the cosmetic agent as claimed in claim 27 and optionally is rinsed with water.
